(19) United States
(12) Patent Application Publication
(10) Pub. No.: US 2007/0117842 A1
(43) Pub. Date: May 24, 2007

(54) Polymorph of 4-[3-chloro-4-(cyclopropylaminocarbonyl)aminophenoxy]-7-methoxy-6- quinolinecarboxamide and a process for the preparation of the same

(76) Inventors: Itaru Arimoto, Tokyo (JP); Kazuhiro Yoshizawa, Ibaraki (JP); Atsushi Kamada, Ibaraki (JP)

Correspondence Address: CHOATE, HALL & STEWART LLP, TWO INTERNATIONAL PLACE, BOSTON, MA, 02110, US

(21) Appl. No.: 10/553,927

(22) PCT Filed: Apr. 22, 2004
(86) PCT No.: PCT/JP04/05788

§ 371 Date: Jun. 30, 2006

Related U.S. Application Data
(60) Provisional application No. 60/464,674, filed on Apr. 22, 2003.

(51) Int. Cl. A61K 31/4704

(52) U.S. Cl. 514/312; 546/156

(57) ABSTRACT

A polymorph (A) of 4-(3-chloro-4-(cyclopropylaminocarbonyl)aminophenoxy)-7-methoxy-6-quinolinecarboxamide, having a diffraction peak at a diffraction angle (2θ±0.2°) of 15.75° in a powder X-ray diffraction; and a polymorph (B) of 4-[3-chloro-4-(cyclopropylaminocarbonyl)aminophenoxy]-7-methoxy-6-quinolinecarboxamide, having a diffraction peak at a diffraction angle (2θ±0.2°) of 21.75° in a powder X-ray diffraction.

### TECHNICAL FIELD OF THE INVENTION

The present invention relates to a polymorph of 4-[3-chloro-4-(cyclopropylaminocarbonyl)aminophenoxy]-7-methoxy-6-quinolinecarboxamide and a process for the preparation of the same.

### BACKGROUND ART

4-[3-chloro-4-(cyclopropylaminocarbonyl)aminophenoxy]-7-methoxy-6-quinolinecarboxamide (additional name: 4-[3-chloro-4-(N′-cyclopropylureido)phenoxy]-7-methoxyquinoline-6-carboxamide) is known to show an excellent angiogenesis inhibitory action (WO 02/32872). 4-[3-chloro-4-(cyclopropylaminocarbonyl)aminophenoxy]-7-methoxy-6-quinolinecarboxamide is also known to show a strong c-Kit kinase inhibitory action (95th Annual Meeting Proceedings, AACR (American Association for Cancer Research), Volume 45, Page 1070-1071, 2004).

### DISCLOSURE OF THE INVENTION

However, for 4-[3-chloro-4-(cyclopropylaminocarbonyl)aminophenoxy]-7-methoxy-6-quinolinecarboxamide, there has been needed crystals of the compound expected to be more excellent in physical properties and stability than those obtained by conventional preparation processes, and a process to prepare the crystals easily and with a high purity.

Thus, an object of the present invention is to provide crystals of 4-[3-chloro-4-(cyclopropylaminocarbonyl)aminophenoxy]-7-methoxy-6-quinolinecarboxamide and a process for the preparation of the crystals.

In order to achieve the above object, the present invention provides polymorphs (1) to (10) below.

(1): A polymorph (A) of 4-(3-chloro-4-(cyclopropylaminocarbonyl)aminophenoxy)-7-methoxy-6-quinolinecarboxamide, having a diffraction peak at a diffraction angle (2θ±0.2°) of 15.75° in a powder X-ray diffraction.

(2): The polymorph (A) according to (1), wherein the polymorph further has diffraction peaks at diffraction angles (2θ±0.2°) of 9.98° and 11.01°in a powder X-ray diffraction.

(3): A polymorph (A) of 4-[3-chloro-4-(cyclopropylaminocarbonyl)aminophenoxy]-7-methoxy-6-quinolinecarboxamide, having an absorption band at a wavenumber of 3452.3±2.5 cm⁻¹ in an infrared absorption spectrum in potassium bromide.

(4): The polymorph (A) according to (1) or (2), wherein the polymorph has an absorption band at a wavenumber of 3452.3±2.5 cm⁻¹ in an infrared absorption spectrum in potassium bromide.

(5): The polymorph (A) according to (3) or (4), wherein the polymorph further has an absorption band at a wavenumber of 1712.2±1.0 cm⁻¹.

(6): A polymorph (B) of 4-[3-chloro-4-(cyclopropylaminocarbonyl)aminophenoxy]-7-methoxy-6-quinolinecarboxamide, having a diffraction peak at a diffraction angle (2θ±0.2°) of 21.75° in a powder X-ray diffraction.

(7): The polymorph (B) according to (6), wherein the polymorph further has diffraction peaks at diffraction angles (2θ±0.2°) of 12.43° and 16.56° in a powder X-ray diffraction.

(8): A polymorph (B) of 4-[3-chloro-4-(cyclopropylaminocarbonyl)aminophenoxy]-7-methoxy-6-quinolinecarboxamide, having an absorption band at a wavenumber of 1557.6±1.0 cm⁻¹ in an infrared absorption spectrum in potassium bromide.

(9): The polymorph (B) according to (6) or (7), wherein the polymorph has an absorption band at a wavenumber of 1557.6±1.0 cm⁻¹ in an infrared absorption spectrum in potassium bromide.

(10): The polymorph (B) according to (8) or (9), wherein the polymorph further has an absorption band at a wavenumber of 1464.4±1.0 cm⁻¹ .

The present invention also provides processes (11) to (28) for preparing a polymorph below.

(11): A process for the preparation of the polymorph (A) of 4-[3-chloro-4-(cyclopropylaminocarbonyl)aminophenoxy]-7-methoxy-6-quinolinecarboxamide according to any one of (1) to (5), comprising a step of dissolving 4-[3-chloro-4-(cyclopropylaminocarbonyl)aminophenoxy]-7-methoxy-6-quinolinecarboxamide, which may be in the form of a crystal or not, in a good organic solvent, followed by rapid admixing with a poor solvent.

(12): A process for the preparation of the polymorph (A) of 4-[3-chloro-4-(cyclopropylaminocarbonyl)aminophenoxy]-7-methoxy-6-quinolinecarboxamide according to any one of (1) to (5), comprising a step of dissolving 4-[3-chloro-4-(cyclopropylaminocarbonyl)aminophenoxy]-7-methoxy-6-quinolinecarboxamide in a good organic solvent with stirring, followed by admixing with a poor solvent in such a way that the resultant crystals precipitate when the stirring is stopped.

(13): A process for the preparation of the polymorph (A) of 4-[3-chloro-4-(cyclopropylaminocarbonyl)aminophenoxy]-7-methoxy-6-quinolinecarboxamide according to any one of (1) to (5), comprising a step of reacting 7-methoxy-4-chloro-quinoline-6-carboxamide with 1-(2-chloro-4-hydroxyphenyl)-3-cyclopropylurea in the presence of a base in a good organic solvent for 4-[3-chloro-4-(cyclopropylaminocarbonyl)aminophenoxy]-7-methoxy-6-quinolinecarboxamide, followed by rapid admixing with a poor solvent.

(14): The process for the preparation according to any one of (11) to (13), wherein the poor solvent is admixed rapidly within 10 minutes.

(15): A process for the preparation of the polymorph (B) of 4-[3-chloro-4-(cyclopropylaminocarbonyl)aminophenoxy]-7-methoxy-6-quinolinecarboxamide according to any one of (6) to (10), comprising a step of dissolving 4-[3-chloro-4-(cyclopropylaminocarbonyl)aminophenoxy]-7-methoxy-6-quinolinecarboxamide, which may be in the form of a salt or not, in a good organic solvent, followed by slow admixing with a poor solvent.

(16): A process for the preparation of the polymorph (B) of 4-[3-chloro-4-(cyclopropylaminocarbonyl)aminophenoxy]-7-methoxy-6-quinolinecarboxamide according to any one of (6) to (10), comprising a step of dissolving 4-[3-chloro-4-(cyclopropylaminocarbonyl)aminophenoxy]-7-methoxy-6-quinolinecarboxamide in a good organic solvent with stirring, followed by admixing with a poor solvent in such a way that the resultant crystals diffuse when the stirring is stopped.

(17): A process for the preparation of the polymorph (B) of 4-[3-chloro-4-(cyclopropylaminocarbonyl)aminophenoxy]-7-methoxy-6-quinolinecarboxamide according to any one of (6) to (10), comprising a step of reacting 7-methoxy-4-chloro-quinoline-6-carboxamide with 1-(2-chloro-4-hydroxyphenyl)-3-cyclopropylurea in the presence of a base in a good organic solvent for 4-[3-chloro-4-(cyclopropylaminocarbonyl)aminophenoxy]-7-methoxy-6-quinolinecarboxamide, followed by slow admixing with a poor solvent.

(18): The process for the preparation according to any one of (15) to (17), wherein the poor solvent is admixed slowly in 1 hour or more.

(19): A process for the preparation of the polymorph (B) of 4-[3-chloro-4-(cyclopropylaminocarbonyl)aminophenoxy]-7-methoxy-6-quinolinecarboxamide according to any one of (6) to (10), comprising a step of heating a polymorph (A) of 4-[3-chloro-4-(cyclopropylaminocarbonyl)aminophenoxy]-7-methoxy-6-quinolinecarboxamide, having a diffraction peak at a diffraction angle (2θ±0.2°) of 15.75° in a powder X-ray diffraction, in suspension in a mixed solvent of a good organic solvent for the polymorph and a poor solvent for the polymorph.

(20): The process for the preparation according to (19), wherein the polymorph (A) is a polymorph further having diffraction peaks at diffraction angles (2θ±0.2°) of 9.98° and 11.01° in a powder X-ray diffraction.

(21): A process for the preparation of the polymorph (B) of 4-[3-chloro-4-(cyclopropylaminocarbonyl)aminophenoxy]-7-methoxy-6-quinolinecarboxamide according to any one of (6) to (10), comprising a step of heating a polymorph (A) of 4-[3-chloro-4-(cyclopropylaminocarbonyl)aminophenoxy]-7-methoxy-6-quinolinecarboxamide, having an absorption band at a wavenumber of 3452.3±2.5 cm⁻¹ in an infrared absorption spectrum in potassium bromide, in suspension in a mixed solvent of a good organic solvent for the polymorph and a poor solvent for the polymorph.

(22): The process for the preparation according to (19) or (20), wherein the polymorph (A) is a polymorph having an absorption band at a wavenumber of 3452.3±2.5 cm⁻¹ in an infrared absorption spectrum in potassium bromide.

(23): The process for the preparation according to (21) or (22), wherein the polymorph (A) is a polymorph further having an absorption band at a wavenumber of 1712.2±1.0 cm⁻¹.

(24): The process for the preparation according to any one of (11) to (23), wherein the good organic solvent is dimethylsulfoxide, dimethylimidazolidinone, 1-methyl-2-pyrrolidinone, N,N-dimethylformamide, N,N-dimethylacetamide, acetic acid, sulforane, or a mixed solvent of at least two of the foregoing.

(25): The process for the preparation according to any one of (11) to (23), wherein the poor solvent is water, acetone, acetonitrile, ethyl acetate, isopropyl acetate, methanol, ethanol, n-propanol, isopropanol, or a mixed solvent of at least two of the foregoing.

(26): The process for the preparation according to (13), (14), (17) or (18), wherein the base is potassium t-butoxide, cesium carbonate or potassium carbonate.

The present invention also provides the followings.

(27): A prophylactic or therapeutic agent for a disease for which angiogenesis inhibition is effective, comprising as an active ingredient, the polymorph according to any one of (1) to (10).

(28): An angiogenesis inhibitor, comprising as an active ingredient, the polymorph according to any one of (1) to (10).

(29): An anti-tumor agent, comprising as an active ingredient, the polymorph according to any one of (1) to (10).

(30): The anti-tumor agent according to (29), wherein the tumor is a pancreatic cancer, a gastric cancer, a colon cancer, a breast cancer, a prostate cancer, a lung cancer, a renal cancer, a brain tumor, a blood cancer or an ovarian cancer.

(31): A therapeutic agent for angioma, comprising as an active ingredient, the polymorph according to any one of (1) to (10).

(32): A cancer metastasis inhibitor, comprising as an active ingredient, the polymorph according to any one of (1) to (10).

(33): A therapeutic agent for retinal neovascularization, comprising as an active ingredient, the polymorph according to any one of (1) to (10).

(34): A therapeutic agent for diabetic retinopathy, comprising as an active ingredient, the polymorph according to any one of (1) to (10).

(35): A therapeutic agent for an inflammatory disease, comprising as an active ingredient, the polymorph according to any one of (1) to (10).

(36): The therapeutic agent for an inflammatory disease according to (35), wherein the inflammatory disease is deformant arthritis, rheumatoid arthritis, psoriasis or delayed hypersensitivity reaction.

(37): A therapeutic agent for atherosclerosis, comprising as an active ingredient, the polymorph according to any one of (1) to (10).

(38): A prophylactic or therapeutic method for a disease for which angiogenesis inhibition is effective, comprising administering to a patient, a pharmacologically effective dose of the polymorph according to any one of (1) to (10).

(39): Use of the polymorph according to any one of (1) to (10) for the manufacture of a prophylactic or therapeutic agent for a disease for which angiogenesis inhibition is effective.

The present invention also provides the followings.

(40): A c-Kit kinase inhibitor comprising as an active ingredient, the polymorph according to any one of (1) to (10).

(41): An anti-cancer agent for treating a cancer expressing excessive c-Kit kinase or a mutant c-Kit kinase, comprising as an active ingredient, the polymorph according to any one of (1) to (10).

(42): The anti-cancer agent according to (41), wherein the cancer expressing excessive c-Kit kinase or a mutant c-Kit kinase is acute myelogenous leukemia, mast cell leukemia, a small cell lung cancer, GIST, a testicular cancer, an ovarian cancer, a breast cancer, a brain cancer, neuroblastoma or a colorectal cancer.

(43): The anti-cancer agent according to (41), wherein the cancer expressing excessive c-Kit kinase or a mutant c-Kit kinase is acute myelogenous leukemia, a small cell lung cancer or GIST.

(44): The anti-cancer agent according to (41), which is applied to a patient for which a cancer expressing excessive c-Kit kinase or a mutant c-Kit kinase is identified.

(45): A therapeutic agent for mastocytosis, allergy or asthma, comprising as an active ingredient, the polymorph according to any one of (1) to (10).

(46): A therapeutic method for a cancer, comprising administering to a patient suffering from a cancer expressing excessive c-Kit kinase or a mutant c-Kit kinase, a pharmacologically effective dose of the polymorph according to any one of (1) to (10).

(47): The method according to (46), wherein the cancer expressing excessive c-Kit kinase or a mutant c-Kit kinase is acute myelogenous leukemia, mast cell leukemia, a small cell lung cancer, GIST, a testicular cancer, an ovarian cancer, a breast cancer, a brain cancer, neuroblastoma or a colorectal cancer.

(48): The method according to (46), wherein the cancer expressing excessive c-Kit kinase or a mutant c-Kit kinase is acute myelogenous leukemia, a small cell lung cancer or GIST.

(49): A therapeutic method for a cancer, comprising the steps of: extracting cancer cells from a patient suffering from a cancer; confirming that the cancer cells are expressing excessive c-Kit kinase or a mutant c-Kit kinase; and

administering to the patient a pharmacologically effective dose of the c-Kit kinase inhibitor according to (40).

(50): A therapeutic method for mastocytosis, allergy or asthma, comprising administering to a patient suffering from the disease, a pharmacologically effective dose of the c-Kit kinase inhibitor according to (40).

(51): A method for inhibiting the c-Kit kinase activity, comprising applying to a cell expressing excessive c-Kit kinase or a mutant c-Kit kinase, a pharmacologically effective dose of the c-Kit kinase inhibitor according to (40).

(52): Use of the c-Kit kinase inhibitor according to (40) for the manufacture of an anti-cancer agent for treating a cancer expressing excessive c-Kit kinase or a mutant c-Kit kinase.

(53): The use according to (52), wherein the cancer expressing excessive c-Kit kinase or a mutant c-Kit kinase is acute myelogenous leukemia, mast cell leukemia, a small cell lung cancer, GIST, a testicular cancer, an ovarian cancer, a breast cancer, a brain cancer, neuroblastoma or a colorectal cancer.

(54): The use according to (52), wherein the cancer expressing excessive c-Kit kinase or a mutant c-Kit kinase is acute myelogenous leukemia, a small cell lung cancer or GIST.

(55): Use of the c-Kit kinase inhibitor according to (40) for the manufacture of a therapeutic agent for mastocytosis, allergy or asthma.

The polymorph (A) according to the invention has such an advantage that filtration is easy after crystallization.

Also, the polymorph (B) according to the invention can be advantageously used to prepare 4-[3-chloro-4-(cyclopropylaminocarbonyl)aminophenoxy]-7-methoxy-6-quinolinecarboxamide with a high purity.

Further, the polymorph (A) has a property that it undergoes crystal transition to the polymorph (B) by suspending the polymorph (A) in a solvent, and the polymorph (B) has an advantage that it can be obtained stably in a production process.

### BRIEF DESCRIPTION OF THE DRAWINGS

FIG. 1 is a figure illustrating a powder X-ray diffraction pattern of the crystals obtained in Example 1a.

FIG. 2 is a figure illustrating a powder X-ray diffraction pattern of the crystals obtained in Example 1b.

FIG. 3 is a figure illustrating a powder X-ray diffraction pattern of the crystals obtained in Example 1c.

FIG. 4 is a figure illustrating a powder X-ray diffraction pattern of the crystals obtained in Example 2a.

FIG. 5 is a figure illustrating a powder X-ray diffraction pattern of the crystals obtained in Example 2b.

FIG. 6 is a figure illustrating a powder X-ray diffraction pattern of the crystals obtained in Example 2c.

FIG. 7 is a figure illustrating an infrared absorption spectrum of the crystals obtained in Example 1a.

FIG. 8 is a figure illustrating an infrared absorption spectrum of the crystals obtained in Example 1b.

FIG. 9 is a figure illustrating an infrared absorption spectrum of the crystals obtained in Example 1c.

FIG. 10 is a figure illustrating an infrared absorption spectrum of the crystals obtained in Example 2a.

FIG. 11 is a figure illustrating an infrared absorption spectrum of the crystals obtained in Example 2b.

FIG. 12 is a figure illustrating an infrared absorption spectrum of the crystals obtained in Example 2c.

FIG. 13 is a figure showing the results of hygroscopicity of the crystals obtained in Example 1d by microbalance method.

FIG. 14 is a figure showing the results of hygroscopicity of the crystals obtained in Example 2d by microbalance method.

FIG. 15 is a figure showing the results of immunoblot of phosphorylated c-Kit kinase by SCF stimulation.

FIG. 16 is a graph showing the relationship between the number of days elapsed after transplantation and tumor volume when H526 was transplanted to a nude mouse.

FIG. 17 is a figure showing the results of the immunoblot of phosphorylated c-Kit kinase, c-Kit kinase and β-actin when H526 was transplanted to a nude mouse.

### BEST MODE FOR CARRYING OUT THE INVENTION

The polymorph (A) of 4-[3-chloro-4-(cyclopropylaminocarbonyl)aminophenoxy]-7-methoxy-6-quinolinecarboxamide of the invention can be produced, for example, by the following method.

4-[3-chloro-4-(cyclopropylaminocarbonyl)aminophenoxy]-7-methoxy-6-quinolinecarboxamide is dissolved in a suitable dissolvable organic solvent (such as dimethylsulfoxide, dimethylimidazolidine, 1-methyl-2-pyrrolidinone, N,N-dimethylformamide, N,N-dimethylacetamide, acetic acid or sulforane), followed by rapid (for example, within 10 minutes) admixing with an undissolvable solvent (such as water, acetone, acetonitrile, ethyl acetate, isopropyl acetate, methanol, ethanol, n-propanol, isopropanol, or a mixed solvent thereof) to produce the polymorph (A). The crystals may appear when the undissolvable solvent is admixed rapidly, and the crystals precipitate in the solvent when the stirring is stopped.

Alternatively, the polymorph (A) can be also obtained by reacting 1-(2-chloro-4-hydroxyphenyl)-3-cyclopropylurea with 7-methoxy-4-chloro-quinoline-6-carboxamide in an organic solvent (such as dimethylsulfoxide (DMSO), dimethylimidazolidinone, 1-methyl-2-pyrrolidinone, N,N-dimethylformamide, N,N-dimethylacetamide, or sulforane) in the presence of a base (such as potassium t-butoxide, cesium carbonate, or potassium carbonate), followed by rapid (for example, within 10 minutes) admixing with an undissolvable solvent (such as water, acetone, acetonitrile, ethyl acetate, isopropyl acetate, methanol, ethanol, n-propanol, isopropanol or a mixed solvent thereof).

More specifically, for example, to a mixture of 1-(2-chloro-4-hydroxyphenyl)-3-cyclopropylurea, 7-methoxy-4-chloro-quinoline-6-carboxamide (1 equivalent or more relative to 1-(2-chloro-4-hydroxyphenyl)-3-cyclopropylurea) and potassium t-butoxide (1 equivalent or more relative to 1-(2-chloro-4-hydroxyphenyl)-3-cyclopropylurea), is added 5- to 10-fold volume of DMSO relative to 1-(2-chloro-4-hydroxyphenyl)-3-cyclopropylurea at room temperature, followed by heating to react at 55-75° C. with stirring for 20 hours or more. To the mixture is added 15-fold volume of an undissolvable solvent (20-50% acetone-water or 20-50% 2-propanol-water) relative to 1-(2-chloro-4-hydroxyphenyl)-3-cyclopropylurea with heating and stirring at 60-65° C. within 8 minutes, then the crystals can appear. Preferably, seed crystals are added when the undissolvable solvent is added in order to allow the crystals to appear. The reaction mixture in which the crystals appeared is stirred at room temperature to 40° C. for 3 hours or more, and the crystals are filtered off to give the polymorph (A).

The polymorph (B) of 4-[3-chloro-4-(cyclopropylaminocarbonyl)aminophenoxy]-7-methoxy-6-quinolinecarboxamide of the invention can be produced, for example, by the following method.

4-[3-chloro-4-(cyclopropylaminocarbonyl)aminophenoxy]-7-methoxy-6-quinolinecarboxamide can be dissolved in a suitable dissolvable organic solvent (such as DMSO, dimethylimidazolidine, 1-methyl-2-pyrrolidinone, N,N-dimethylformamide, N,N-dimethylacetamide, acetic acid, or sulforane), followed by slow (for example, for 1 hour or more) admixing with an undissolvable solvent (such as water, acetone, acetonitrile, ethyl acetate, isopropyl acetate, methanol, ethanol, n-propanol, isopropanol, or a mixed solvent thereof) to produce the polymorph (B). The crystals may appear when the undissolvable solvent is mixed slowly, and the crystals diffuse in the whole solvent when the stirring is stopped.

More specifically, for example, to 4-[3-chloro-4-(cyclopropylaminocarbonyl)aminophenoxy]-7-methoxy-6-quinolinecarboxamide is added 4- to 5-fold volume of a dissolvable solvent (DMSO or 1-methyl-2-pyrrolidinone) relative to 4-[3-chloro-4-(cyclopropylaminocarbonyl)aminophenoxy]-7-methoxy-6-quinolinecarboxamide, followed by heating and stirring at 80° C. or more to dissolve the compound. To the reaction mixture is added 10- to 20-fold volume of an undissolvable solvent (isopropyl acetate, ethyl acetate, methanol, or isopropanol) relative to 4-[3-chloro-4-(cyclopropylaminocarbonyl)aminophenoxy]-7-methoxy-6-quinolinecarboxamide over 30 minutes or more with heating and stirring at 65-85° C., then the crystals can appear. Preferably, seed crystals are added when the undissolvable solvent is added in order to allow the crystals to appear. The reaction mixture in which the crystals appeared is heated and stirred at 70° C. or higher for 30 minutes or more and further stirred at room temperature, and the crystals are filtered off to give the polymorph (B).

The polymorph (B) can be also produced by heating and suspending the polymorph (A) of 4-[3-chloro-4-(cyclopropylaminocarbonyl)aminophenoxy]-7-methoxy-6-quinolinecarboxamide in a mixed solvent of a dissolvable solvent and an undissolvable solvent.

Alternatively, the polymorph (B) can be also obtained by reacting 1-(2-chloro-4-hydroxyphenyl)-3-cyclopropylurea with 7-methoxy-4-chloro-quinoline-6-carboxamide in an organic solvent (such as DMSO, dimethylimidazolidinone, 1-methyl-2-pyrrolidinone, N,N-dimethylformamide, N,N-dimethylacetamide, or sulforane) in the presence of a base (such as potassium t-butoxide, cesium carbonate, or potassium carbonate), followed by slow (for example, for 30 minutes or more) admixing with an undissolvable solvent (such as water, acetone, acetonitrile, ethyl acetate, isopropyl acetate, methanol, ethanol, n-propanol, isopropanol, or a mixed solvent thereof).

More specifically, for example, to a mixture of 1-(2-chloro-4-hydroxyphenyl)-3-cyclopropylurea, 7-methoxy-4-chloro-quinoline-6-carboxamide (1 equivalent or more relative to 1-(2-chloro-4-hydroxyphenyl)-3-cyclopropylurea) and potassium t-butoxide (1 equivalent or more relative to 1-(2-chloro-4-hydroxyphenyl)-3-cyclopropylurea), is added 5- to 10-fold volume of DMSO relative to 1-(2-chloro-4-hydroxyphenyl)-3-cyclopropylurea at room temperature, followed by heating to react at 55-75° C. with stirring for 20 hours or more. To the mixture is added 15-fold volume of an undissolvable solvent (33% acetone-water) relative to 1-(2-chloro-4-hydroxyphenyl)-3-cyclopropylurea with heating and stirring at 60-65° C. for 2 hours or more, and the crystals can appear. The reaction mixture in which the crystals appeared is heated and stirred at 40° C. for 3 hours or more, and the crystals are filtered off to give the polymorph (B).

The dosage of a medicine according to the invention will differ depending on the severity of symptoms, patient age, gender and weight, administration form and type of disease, but administration may usually be from 100 μg to 10 g per day for adults, either at once or in divided doses.

There are no particular restrictions on the form of administration of a medicine according to the invention, and it may usually be administered orally or parenterally by conventional methods.

Common excipients, binders, glossy agents, coloring agents, taste correctors and the like, and if necessary stabilizers, emulsifiers, absorption promoters, surfactants and the like, may also be used for formulation, with inclusion of components ordinarily used as starting materials for formulation of pharmaceutical preparations by common methods.

Examples of such components which may be used include animal and vegetable oils (soybean oil, beef tallow, synthetic glycerides, etc.), hydrocarbons (liquid paraffin, squalane, solid paraffin, etc.), ester oils (octyldodecyl myristate, isopropyl myristate, etc.), higher alcohols (cetostearyl alcohol, behenyl alcohol, etc.), silicone resins, silicone oils, surfactants (polyoxyethylene fatty acid esters, sorbitan fatty acid esters, glycerin fatty acid esters, polyoxyethylenesorbitan fatty acid esters, polyoxyethylene hydrogenated castor oil, polyoxyethylenepolyoxypropylene block copolymer, etc.), water-soluble polymers (hydroxyethyl cellulose, polyacrylic acid, carboxyvinyl polymer, polyethyleneglycol, polyvinylpyrrolidone, methyl cellulose, etc.), alcohols (ethanol, isopropanol, etc.), polyhydric alcohols (glycerin, propyleneglycol, dipropyleneglycol, sorbitol, etc.), sugars (glucose, sucrose, etc.), inorganic powders (silicic anhydride, aluminium magnesium silicate, aluminium silicate, etc.), purified water and the like. For pH adjustment there may be used inorganic acids (hydrochloric acid, phosphoric acid, etc.), alkali metal salts of inorganic acids (sodium phosphate, etc.), inorganic bases (sodium hydroxide, etc.), organic acids (lower fatty acids, citric acid, lactic acid, etc.), alkali metal salts of organic acids (sodium citrate, sodium lactate, etc.), and organic bases (arginine, ethanolamine, etc.). If necessary, preservatives, antioxidants and the like may also be added.

### EXAMPLES

The present invention will be explained through the following examples, but these examples are in no way limitative on the invention.

### Preparation Example 1

### Preparation of 1-(2-chloro-4-hydroxyphenyl)-3-cyclopropylurea

### a) Phenyl N-(2-chloro-4-hydroxyphenyl)carbamate

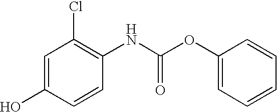

To a suspension of 4-amino-3-chlorophenol (23.7 g) suspended in N,N-dimethylformamide (100 mL) was added pyridine (23.4 mL) while cooling in an ice bath, and phenyl chlorocarbonate (23.2 mL) was added dropwise below 20° C. After stirring at room temperature for 30 minutes, water (400 mL), ethyl acetate (300 mL), and 6N-HCl (48 mL) were added and stirred, and the organic phase was separated off. The organic phase was washed twice with a 10% aqueous sodium chloride solution (200 mL), and dried over magnesium sulfate. The solvent was evaporated to give 46 g of the titled compound as a solid.

¹H-NMR (CDCl₃): 5.12 (1h, br s), 6.75 (1H, dd, J=9.2, 2.8 Hz), 6.92 (1H, d, J=2.8 Hz), 7.18-7.28 (4H, m), 7.37-7.43 (2H, m), 7.94 (1H, br s).

### b) 1-(2-chloro-4-hydroxypenyl)-3-cyclopropylurea

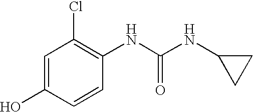

To a solution of phenyl N-(2-chloro-4-hydroxyphenyl)carbamate in N,N-dimethylformamide (100 mL) was added cyclopropylamine (22.7 mL) with cooling in an ice bath, and the stirring was continued at room temperature overnight. Water (400 mL), ethyl acetate (300 mL), and 6N-HCl (55 mL) were added thereto, the mixture was stirred, and the organic phase was separated off. The organic phase was washed twice with a 10% aqueous sodium chloride solution (200 mL), and dried over magnesium sulfate. The solvent was evaporated to give prism crystals, which were filtered off and washed with heptane to give 22.8 g of the titled compound (yield from 4-amino-3-chlorophenol: 77%).

¹H-NMR (CDCl₃): 0.72-0.77 (2H, m), 0.87-0.95 (2H, m), 2.60-2.65 (1H, m), 4.89 (1H, br s), 5.60 (1H, br s), 6.71 (1H, dd, J=8.8, 2.8 Hz), 6.88 (1H, d, J=2.8 Hz), 7.24-7.30 (1H, br s), 7.90 (1H, d, J=8.8H).

### Preparation Example 2

### Preparation of 7-methoxy-4-chloro-quinoline-6-carboxamide

### a) 4-[(2,2-dimethyl-4,6-dioxo-[1,3]dioxane-5-ylidenemethyl)-amino]-2-methoxybenzoic acid ethyl ester

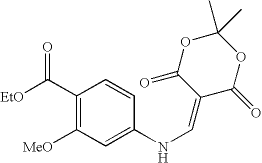

To a suspension of 4-amino-2-methoxybenzoic acid ethyl ester (CAS NO. 14814-06-3) (3.00 g) suspended in 2-propanol (15 mL) were added Meldrum's acid (2.44 g: 1.1 equivalent weight) and ethyl orthoformate (7.5 mL), followed by heating at 85° C. for 1 hour. The resultant precipitates were filtered off and washed with MTBE (methyl-tert-butylether) to give 4.92 g of titled compound (yield: 81%).

¹H-NMR (DMSO-d₆): 1.26 (3H, t, J=7.0 Hz), 1.60 (6H, s), 3.85 (3H, s), 4.20 (2H, q, J=7.0 Hz), 7.15 (1H, br d, J=8.4 Hz), 7.38 (1H, s), 7.69 (1H, d, J=8.4 Hz), 8.63 (1H, s).

### b) 7-methoxy-4-oxo-1,4-dihydroquinoline-6-carboxylic acid ethyl ester

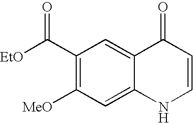

4-[(2,2-dimethyl-4,6-dioxo-[1,3]dioxane-5-ylidenemethyl)-amino]-2-methoxybenzoic acid ethyl ester (3.55 g) was suspended in Dawtherm (10.7 mL), and the suspension was heated in an oil bath at 200° C. for 50 minutes. After allowed to stand at room temperature, MTBE (10 mL) was added thereto, then the resultant precipitates were filtered off and dried under vacuum to give 1.56 g of the titled compound (yield: 63%).

¹H-NMR (DMSO-d₆): 1.29 (3H, t, J=7.2 Hz), 3.87 (3H, s), 4.25 (2H, q, J=7.2 Hz), 5.79 (1H, d, J=7.4 Hz), 7.01 (1H, s), 7.84 (1H, d, J=7.4 Hz), 8.38 (1H, s), 11.77 (1H, br s).

### c) 7-methoxy-4-oxo-1,4-dihydroquinoline-6-carboxylic acid

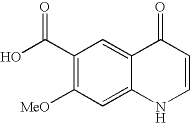

To a solution of 7-methoxy-4-oxo-1,4-dihydroquinone-6-carboxylic acid ethyl ester (120 mg) dissolved in ethanol (1 mL) was added a 25% aqueous sodium hydroxide solution (0.2 mL), and the stirring was continued at 65° C. for 1 hour. 6N-HCl (0.5 mL) was added thereto, then the resultant precipitates were filtered off, washed with water, and dried under vacuum to give 100 mg of the titled compound (yield: 94%).

¹H-NMR (DMSO-d₆): 4.87 (3H, s), 6.14 (1H, d, J=7.4 Hz), 7.04 (1H, s), 7.98 (1H, d, J=6.0 Hz), 8.40 (1H, s).

### d) 7-methoxy-4-chloro-quinoline-6-carboxamide

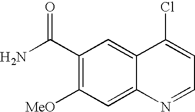

To 7-methoxy-4-oxo-1,4-dihydroquinoline-6-carboxylic acid (2.0 g) were added thionyl chloride (10 mL) and a small amount of N,N-dimethylformamide, and the mixture was heated under reflux for 2 hours. The mixture was concentrated under vacuum, followed by azeotropic distillation twice with toluene to give 7-methoxy-4-chloro-quinoline-6-carbonyl chloride (2.7 g).

Subsequently, 7-methoxy-4-chloro-quinoline-6-carbonyl chloride (2.7 g) thus obtained was dissolved in tetrahydrofuran (150 mL), and the solution was cooled to 0° C. 30% aqueous ammonia (5 mL) was added thereto, and the mixture was stirred at room temperature for 30 minutes. Water was added thereto, and the resultant mixture was extracted three times with ethyl acetate. The combined organic phase was washed with water and saturated brine, dried over sodium sulfate, and dried under vacuum to give the titled compound (1.35 g).

¹H-NMR (DMSO-d₆): 4.03 (3H, s), 7.56-7.66 (2H, m), 7.79 (1H, brs), 7.88 (1H, brs), 8.46-8.49 (1H, m), 8.78-8.82 (1H, m).

### Preparation Example 3

### Preparation of 4-(3-chloro-4-(cyclopropylaminocarbonyl)aminophenoxy)-7-methoxy-6-quinolinecarboxamide

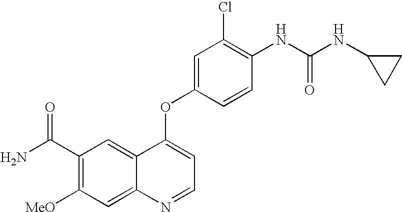

To DMSO (20 mL) were added 7-methoxy-4-chloro-quinoline-6-carboxamide (0.983 g), 1-(2-chloro-4-hydroxyphenyl)-3-cyclopropylurea (1.13 g) and cesium carbonate (2.71 g), and the mixture was heated and stirred at 70° C. for 23 hours. The reaction mixture was cooled to room temperature, water (50 mL) was added, and the resultant solid was then filtered off to give 1.56 g of the titled compound (yield: 88%).

¹H-NMR (d₆-DMSO): 0.41 (2H, m), 0.66 (2H, m), 2.56 (1H, m), 4.01 (3H, s), 6.51 (1H, d, J=5.6 Hz), 7.18 (1H, d, J=2.8 Hz), 7.23 (1H, dd, J=2.8, 8.8 Hz), 7.48 (1H, d, J=2.8 Hz), 7.50 (1H, s), 7.72 (1H, s), 7.84 (1H, s), 7.97 (1H, s), 8.25 (1H, d, J=8.8 Hz), 8.64 (1H, s), 8.65 (1H, d, J=5.6 Hz).

### Example 1a

### Preparation of polymorph (A) of 4-(3-chloro-4-(cyclopropylaminocarbonyl)aminophenoxy)-7-methoxy-6-quinolinecarboxamide

Firstly, 1-(2-chloro-4-hydroxyphenyl)-3-cyclopropylurea was obtained in a similar manner as Preparation Example 1, and 7-methoxy-4-chloro-quinoline-6-carboxamide was obtained in a similar manner as Preparation Example 2.

Then, to a mixture of 1-(2-chloro-4-hydroxyphenyl)-3-cyclopropylurea (114.9 g), 7-methoxy-4-chloro-quinoline-6-carboxamide (80.0 g) and potassium t-butoxide (56.9 g) was added DMSO (800 mL) at room temperature, and the mixture was heated and stirred at 55° C. for 20 hours and, then further at 60° C. for 4 hours. To the reaction mixture, 33% (v/v) acetone-water (165 mL) was added in 1 minute at 60° C. with stirring. Additional 33% (v/v) acetone water (1035 mL) was added dropwise over 7 minutes to allow the crystals to appear, followed by stirring at 40° C. for 19 hours. The crystals were filtered off, washed with 33% (v/v) acetone-water and acetone, and dried to give 131.9 g of yellowish brown granular crystal (the polymorph (A)).

### Examples 1b, 1c and 1d

The polymorph (A) of 4-(3-chloro-4-(cyclopropylaminocarbonyl)aminophenoxy)-7-methoxy-6-quinolinecarboxamide was obtained in a similar manner as Example 1a.

### Example 2a

### Preparation of polymorph (B) of 4-(3-chloro-4-(cyclopropylaminocarbonyl)aminophenoxy)-7-methoxy-6-quinolinecarboxamide

Firstly, 1-(2-chloro-4-hydroxyphenyl)-3-cyclopropylurea was obtained in a similar manner as Preparation Example 1, and 7-methoxy-4-chloro-quinoline-6-carboxamide was obtained in a similar manner as Preparation Example 2.

Secondly, to a mixture of 1-(2-chloro-4-hydroxyphenyl)-3-cyclopropylurea (11.49 g), 7-methoxy-4-chloroquinoline-6-carboxamide (8.00 g) and potassium t-butoxide (5.69 g) was added DMSO (80 mL) at room temperature, and the mixture was heated and stirred at 60° C. for 25 hours. The reaction mixture was divided into four equal parts. To an aliquot was added dropwise 33% (v/v) acetone-water (10 mL) over 3 hours at 60° C. with stirring to allow the crystals to appear. Additional 33% (v/v) acetone-water (20 mL) was added dropwise over 1 hour, and the stirring was continued at 40° C. for 5 hours. The resultant crystals were filtered off, washed with 33% (v/v) acetone-water and acetone, and dried to give 3.22 g of white fibrous crystals (the polymorph (B)).

### Examples 2b, 2c and 2d

A polymorph (B) of 4-(3-chloro-4-(cyclopropylaminocarbonyl)aminophenoxy)-7-methoxy-6-quinolinecarboxamide was obtained in a similar manner as Example 2a.

### Example 3

### Preparation of polymorph (B) of 4-(3-chloro-4-(cyclopropylaminocarbonyl)aminophenoxy)-7-methoxy-6-quinolinecarboxamide

Firstly, 4-(3-chloro-4-(cyclopropylaminocarbonyl)aminophenoxy)-7-methoxy-6-quinolinecarboxamide was obtained in a similar manner as Preparation Example 3.

Secondly, the resultant 4-(3-chloro-4-(cyclopropylaminocarbonyl)aminophenoxy)-7-methoxy-6-quinolinecarboxamide (42.7 g) was added to 1,3-dimethyl-2-imidazolidinone (425 mL) to dissolve at 84° C., and then isopropyl acetate (1000 mL) was added over 20 minutes. After stirring at 80° C. for 30 minutes and further at room temperature for 6 hours, the crystals were filtered off to give 41.1 g of the polymorph (B).

### Example 4

### Crystal Transition from the Polymorph (A) to the Polymorph (B)

To a mixed solvent of DMSO (1.7 mL) and 33% (v/v) acetone water (0.17, 0.34, 0.51 or 0.85 mL) was added 300 mg of the polymorph (A) of 4-(3-chloro-4-(cyclopropylaminocarbonyl)aminophenoxy)-7-methoxy-6-quinolinecarboxamide, and the mixture was heated and stirred at 60° C. for 3 hours, during which 4-(3-chloro-4-(cyclopropylaminocarbonyl)aminophenoxy)-7-methoxy-6-quinolinecarboxamide did not dissolve and remained in suspension.

These suspensions were filtered to collect 4-(3-chloro-4-(cyclopropylaminocarbonyl)aminophenoxy)-7-methoxy-6-quinolinecarboxamide (184 to 266 mg). Evaluation of the forms of the resultant crystals demonstrated that crystal transition to the polymorph (B) occurred in every case.

In this connection, when 300 mg of the polymorph (A) was dissolved in DMSO (1.7 mL) followed by heating and stirring at 60° C. for 3 hours without adding 33% acetone-water, most of the polymorph (A) dissolved.

### Comparative Example 1

### Crystal Transition from the Polymorph (B) to the Polymorph (A)

To a mixed solvent of DMSO (1.7 mL) and 33% (v/v) acetone-water (0.17, 0.34, 0.51 or 0.85 mL), was added 300 mg of the polymorph (B) of 4-(3-chloro-4-(cyclopropylaminocarbonyl)aminophenoxy)-7-methoxy-6-quinolinecarboxamide, and the mixture was heated and stirred at 60° C. for 3 hours, during which the 4-(3-chloro-4-(cyclopropylaminocarbonyl)aminophenoxy)-7-methoxy-6-quinolinecarboxamide did not dissolve and remained in suspension.

These suspensions were filtered to collect 4-(3-chloro-4-(cyclopropylaminocarbonyl)aminophenoxy)-7-methoxy-6-quinolinecarboxamide (141 to 256 mg). Evaluation of the forms of the resultant crystals demonstrated that all of them remained the polymorph (B) to reveal that the transition from the polymorph (B) to the polymorph (A) does not occur under the aforementioned conditions.

In this connection, when 300 mg of the polymorph (B) was dissolved in DMSO (1.7 mL) followed by heating and stirring at 60° C. for 3 hours without adding 33% acetone-water, most of the polymorph (B) dissolved.

(Powder X-ray Diffraction Measurement)

Powder X-ray diffraction analysis of the crystals obtained in respective Examples was carried out according to the powder X-ray diffraction method as described in the Japanese Pharmacopoeia, General Tests under the following measurement conditions using about 100 mg of sample.

Apparatus: Geiger Flex RAD-3C manufactured by Rigaku Denki KK

X-ray: CuKα ray

Counter: Scintillation counter

Filter: monochromatic

Goniometer: horizontal goniometer

Applied Voltage: 40 kV

Charging current: 20 mA

Scan speed: 3°/min

Scan axis: 2θ

Scan range: 2θ=5-30°

Divergent slit: 1°

Scattering slit: 1°

Receiving slit: 0.15 mm

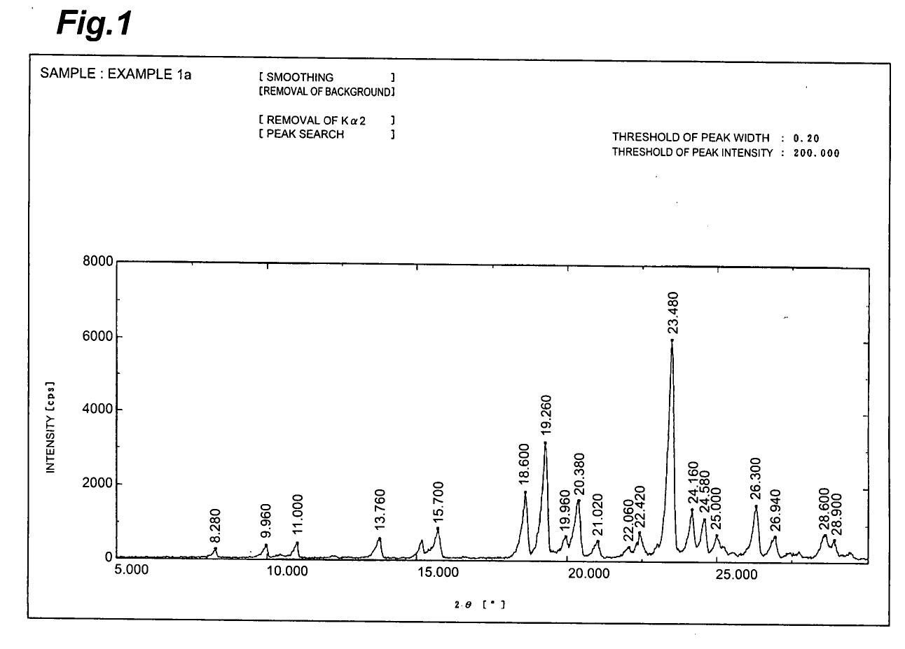

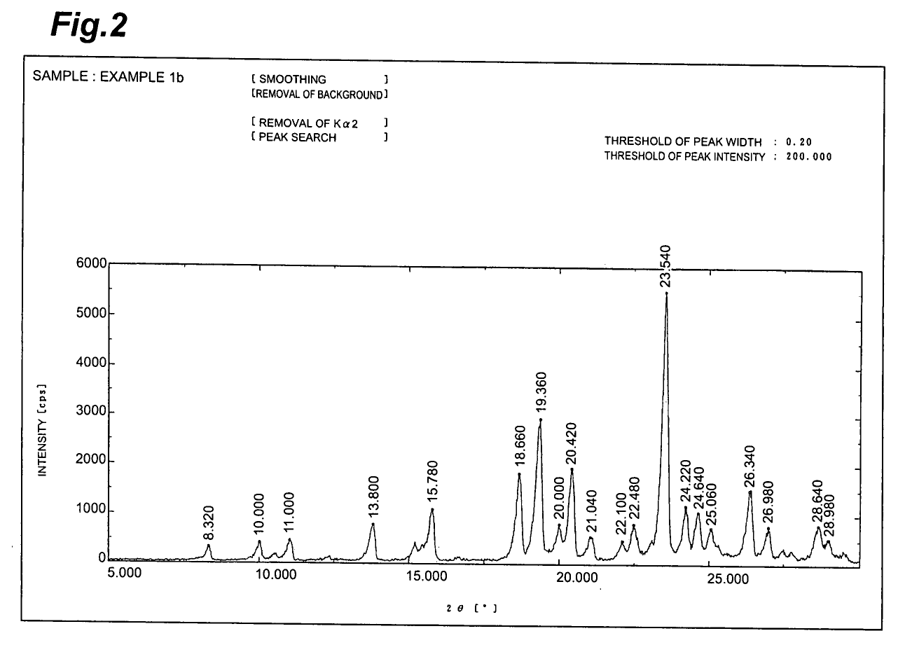

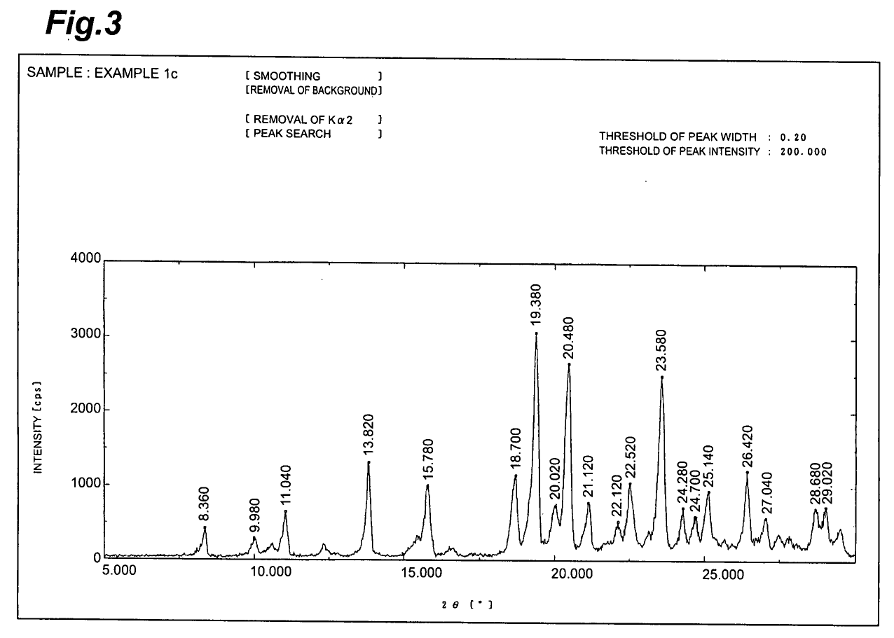

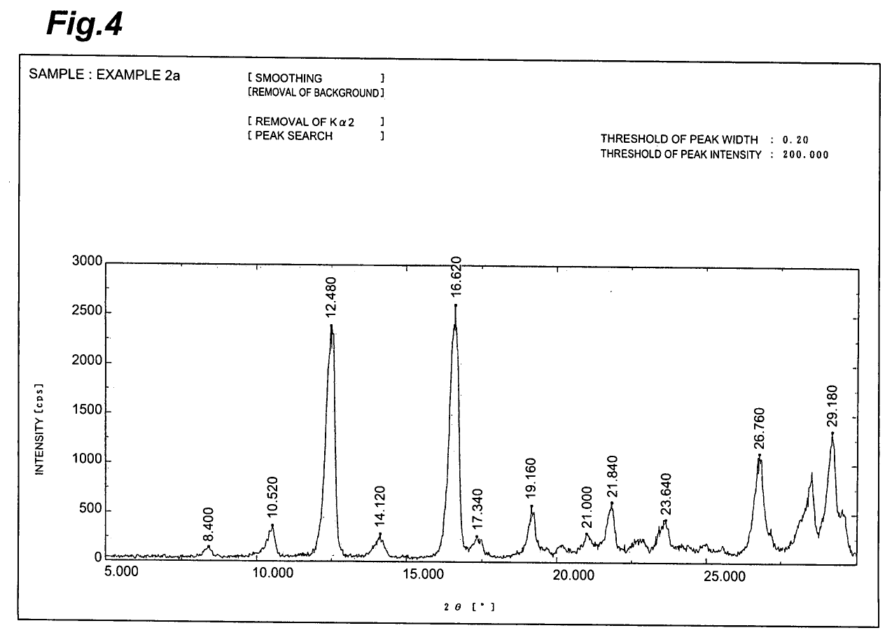

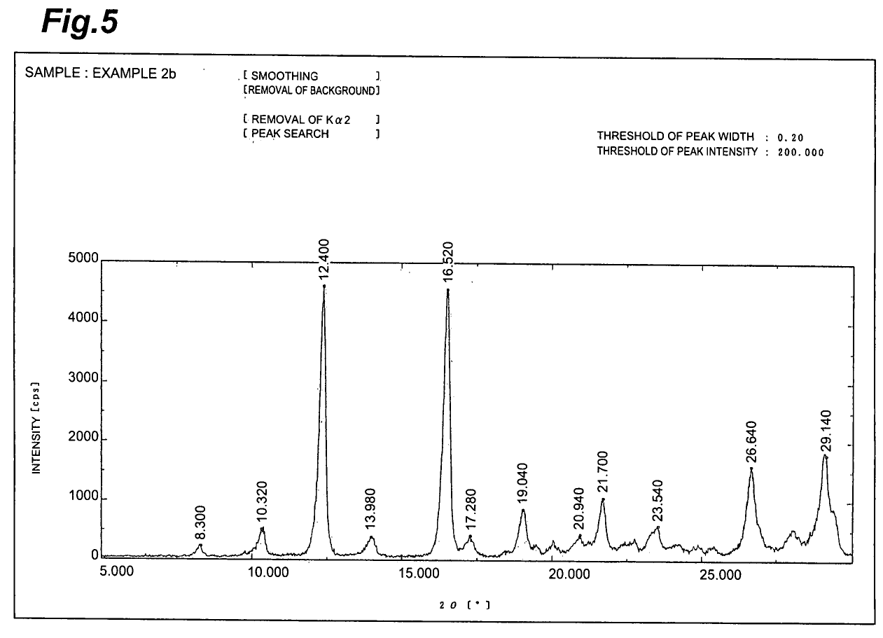

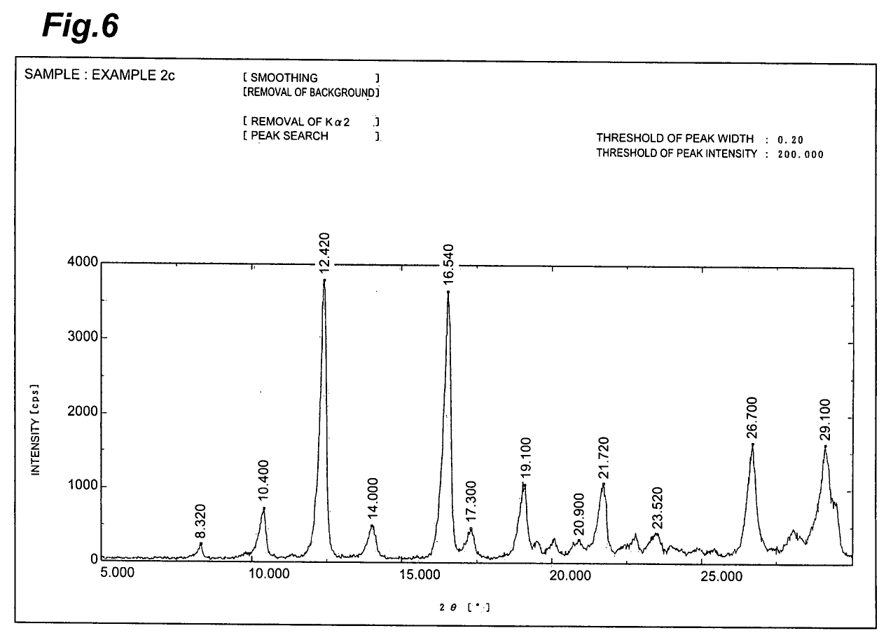

The powder X-ray diffraction patterns of the crystals obtained in Examples 1a-1c and 2a-2c are shown in FIG. 1-6, and peaks of the Diffraction angles (2θ) and intensities are shown in Tables 1-6. Further, the peaks of the diffraction angles (2θ) in respective Examples and the average values of the peaks are listed in Table 7.

TABLE 1
SAMPLE: EXAMPLE 1a

| PEAK | | HALF | | | RELATIVE |
|---|---|---|---|---|---|
| NUMBER | 2θ | WIDTH | D-VALUE | INTENSITY | INTENSITY |
| 1 | 8.280 | ***** | 10.6696 | 290 | 5 |
| 2 | 9.960 | ***** | 8.8734 | 385 | 6 |
| 3 | 11.000 | ***** | 8.0367 | 445 | 7 |
| 4 | 13.760 | ***** | 6.4302 | 582 | 10 |
| 5 | 15.700 | ***** | 6.6398 | 872 | 14 |
| 6 | 18.600 | ***** | 4.7665 | 1860 | 31 |
| 7 | 19.260 | ***** | 4.6046 | 3182 | 53 |
| 8 | 19.960 | ***** | 4.4447 | 678 | 11 |
| 9 | 20.380 | ***** | 4.3540 | 1642 | 27 |
| 10 | 21.020 | ***** | 4.2229 | 552 | 9 |
| 11 | 22.060 | ***** | 4.0261 | 398 | 7 |
| 12 | 22.420 | ***** | 3.9622 | 800 | 13 |
| 13 | 23.480 | ***** | 3.7857 | 6032 | 100 |
| 14 | 24.160 | ***** | 3.6807 | 1432 | 24 |
| 15 | 24.580 | ***** | 3.6187 | 1170 | 19 |
| 16 | 25.000 | ***** | 3.5589 | 738 | 12 |
| 17 | 26.300 | ***** | 3.3858 | 1528 | 26 |
| 18 | 26.940 | ***** | 3.3068 | 705 | 12 |
| 19 | 28.600 | ***** | 3.1186 | 772 | 13 |
| 20 | 28.900 | ***** | 3.0869 | 628 | 10 |

TABLE 2
SAMPLE: EXAMPLE 1b

| PEAK | | HALF | | | RELATIVE |
|---|---|---|---|---|---|
| NUMBER | 2θ | WIDTH | D-VALUE | INTENSITY | INTENSITY |
| 1 | 8.320 | ***** | 10.6184 | 322 | 6 |
| 2 | 10.000 | ***** | 8.8380 | 418 | 8 |
| 3 | 11.000 | ***** | 8.0367 | 458 | 8 |
| 4 | 13.800 | ***** | 6.4117 | 792 | 14 |
| 5 | 15.780 | ***** | 5.6114 | 1095 | 20 |
| 6 | 18.660 | ***** | 4.7513 | 1822 | 33 |
| 7 | 19.360 | ***** | 4.5810 | 2932 | 53 |
| 8 | 20.000 | ***** | 4.4359 | 808 | 15 |
| 9 | 20.420 | ***** | 4.3456 | 1932 | 35 |
| 10 | 21.040 | ***** | 4.2189 | 558 | 10 |
| 11 | 22.100 | ***** | 4.0189 | 480 | 9 |
| 12 | 22.480 | ***** | 3.9518 | 820 | 15 |
| 13 | 23.540 | ***** | 3.7762 | 5522 | 100 |
| 14 | 24.220 | ***** | 3.6717 | 1185 | 21 |
| 15 | 24.640 | ***** | 3.6100 | 1062 | 19 |
| 16 | 25.060 | ***** | 3.5505 | 745 | 13 |
| 17 | 26.340 | ***** | 3.3808 | 1502 | 27 |
| 18 | 26.980 | ***** | 3.3020 | 780 | 14 |
| 19 | 28.640 | ***** | 3.1143 | 810 | 15 |
| 20 | 28.980 | ***** | 3.0785 | 525 | 10 |

TABLE 3
SAMPLE: EXAMPLE 1c

| PEAK | | HALF | | | RELATIVE |
|---|---|---|---|---|---|
| NUMBER | 2θ | WIDTH | D-VALUE | INTENSITY | INTENSITY |
| 1 | 8.360 | ***** | 10.5677 | 425 | 14 |
| 2 | 9.980 | ***** | 8.8556 | 292 | 10 |
| 3 | 11.040 | ***** | 8.0076 | 650 | 21 |
| 4 | 13.820 | ***** | 6.4025 | 1318 | 43 |
| 5 | 15.780 | ***** | 5.6114 | 995 | 32 |
| 6 | 18.700 | ***** | 4.7412 | 1150 | 37 |
| 7 | 19.380 | ***** | 4.5764 | 3075 | 100 |
| 8 | 20.020 | ***** | 4.4315 | 738 | 24 |
| 9 | 20.480 | ***** | 4.3330 | 2658 | 86 |
| 10 | 21.120 | ***** | 4.2031 | 782 | 25 |
| 11 | 22.120 | ***** | 4.0153 | 528 | 17 |
| 12 | 22.520 | ***** | 3.9449 | 1048 | 34 |
| 13 | 23.580 | ***** | 3.7699 | 2492 | 81 |
| 14 | 24.280 | ***** | 3.6628 | 718 | 23 |
| 15 | 24.700 | ***** | 3.6014 | 595 | 19 |
| 16 | 25.140 | ***** | 3.5394 | 940 | 31 |
| 17 | 26.420 | ***** | 3.3707 | 1215 | 40 |
| 18 | 27.040 | ***** | 3.2948 | 582 | 19 |
| 19 | 28.680 | ***** | 3.1100 | 710 | 23 |
| 20 | 29.020 | ***** | 3.0744 | 740 | 24 |

TABLE 4
SAMPLE: EXAMPLE 2a

| PEAK | | HALF | | | RELATIVE |
|---|---|---|---|---|---|
| NUMBER | 2θ | WIDTH | D-VALUE | INTENSITY | INTENSITY |
| 1 | 8.400 | ***** | 10.5175 | 142 | 5 |
| 2 | 10.520 | ***** | 8.4023 | 362 | 14 |
| 3 | 12.480 | ***** | 7.0867 | 2390 | 92 |
| 4 | 14.120 | ***** | 6.2671 | 282 | 11 |
| 5 | 16.620 | ***** | 5.3296 | 2600 | 100 |
| 6 | 17.340 | ***** | 5.1099 | 262 | 10 |
| 7 | 19.160 | ***** | 4.6284 | 572 | 22 |
| 8 | 21.000 | ***** | 4.2268 | 295 | 11 |
| 9 | 21.840 | ***** | 4.0661 | 612 | 24 |
| 10 | 23.640 | ***** | 3.7604 | 440 | 17 |
| 11 | 26.760 | ***** | 3.3287 | 1112 | 43 |
| 12 | 29.180 | ***** | 3.0579 | 1340 | 52 |

TABLE 5
SAMPLE: EXAMPLE 2b

| PEAK | | HALF | | | RELATIVE |
|---|---|---|---|---|---|
| NUMBER | 2θ | WIDTH | D-VALUE | INTENSITY | INTENSITY |
| 1 | 8.300 | ***** | 10.6440 | 228 | 6 |
| 2 | 10.320 | ***** | 8.5646 | 510 | 11 |
| 3 | 12.400 | ***** | 7.1323 | 4600 | 100 |
| 4 | 13.980 | ***** | 6.3295 | 388 | 8 |
| 5 | 16.520 | ***** | 5.3616 | 4555 | 99 |
| 6 | 17.280 | ***** | 5.1275 | 410 | 9 |
| 7 | 19.040 | ***** | 4.6573 | 852 | 19 |
| 8 | 20.940 | ***** | 4.2388 | 432 | 9 |
| 9 | 21.700 | ***** | 4.0920 | 1050 | 23 |
| 10 | 23.540 | ***** | 3.7762 | 585 | 13 |
| 11 | 26.640 | ***** | 3.3434 | 1592 | 35 |
| 12 | 29.140 | ***** | 3.0620 | 1785 | 39 |

TABLE 6
SAMPLE: EXAMPLE 2c

| PEAK | | HALF | | | RELATIVE |
|---|---|---|---|---|---|
| NUMBER | 2θ | WIDTH | D-VALUE | INTENSITY | INTENSITY |
| 1 | 8.320 | ***** | 10.6184 | 240 | 6 |
| 2 | 10.400 | ***** | 8.4989 | 722 | 19 |
| 3 | 12.420 | ***** | 7.1208 | 3788 | 100 |
| 4 | 14.000 | ***** | 6.3205 | 492 | 13 |
| 5 | 16.540 | ***** | 5.3552 | 3642 | 96 |
| 6 | 17.300 | ***** | 5.1216 | 465 | 12 |
| 7 | 19.100 | ***** | 4.6428 | 1052 | 28 |
| 8 | 20.900 | ***** | 4.2468 | 318 | 8 |
| 9 | 21.720 | ***** | 4.0883 | 1078 | 28 |
| 10 | 23.520 | ***** | 3.7794 | 405 | 11 |
| 11 | 26.700 | ***** | 3.3360 | 1628 | 43 |
| 12 | 29.100 | ***** | 3.0661 | 1608 | 42 |

TABLE 7

| Polymorph (A), | | | | Polymorph (B), | | | |
|---|---|---|---|---|---|---|---|
| diffraction angle | | | | diffraction angle | | | |
| Ex. 1a | Ex. 1b | Ex. 1c | Ave. | Ex. 2a | Ex. 2b | Ex. 2c | Ave. |
| 8.28 | 8.32 | 8.36 | 8.32 | 8.40 | 8.30 | 8.32 | 8.34 |
| 9.96 | 10.00 | 9.98 | 9.98 | 10.52 | 10.32 | 10.40 | 10.41 |
| 11.00 | 11.00 | 11.04 | 11.01 | 12.48 | 12.40 | 12.42 | 12.43 |
| 13.76 | 13.80 | 13.82 | 13.79 | 14.12 | 13.98 | 14.00 | 14.03 |
| 15.70 | 15.78 | 15.78 | 15.75 | 16.62 | 16.52 | 16.54 | 16.56 |
| 18.60 | 18.66 | 18.70 | 18.65 | 17.34 | 17.28 | 17.30 | 17.31 |
| 19.26 | 19.36 | 19.38 | 19.33 | 19.16 | 19.04 | 19.10 | 19.10 |
| 19.96 | 20.00 | 20.02 | 19.99 | 21.00 | 20.94 | 20.90 | 20.95 |
| 20.38 | 20.42 | 20.48 | 20.43 | 21.84 | 21.70 | 21.72 | 21.75 |
| 21.02 | 21.04 | 21.12 | 21.06 | 23.64 | 23.54 | 23.52 | 23.57 |
| 22.06 | 22.10 | 22.12 | 22.09 | 26.76 | 26.64 | 26.70 | 26.70 |
| 22.42 | 22.48 | 22.52 | 22.47 | 29.18 | 29.14 | 29.10 | 29.14 |
| 23.48 | 23.54 | 23.58 | 23.53 | | | | |
| 24.16 | 24.22 | 24.28 | 24.22 | | | | |
| 24.58 | 24.64 | 24.70 | 24.64 | | | | |
| 25.00 | 25.06 | 25.14 | 25.07 | | | | |
| 26.30 | 26.34 | 26.42 | 26.35 | | | | |
| 26.94 | 26.98 | 27.04 | 27.99 | | | | |
| 28.60 | 28.64 | 28.68 | 28.64 | | | | |
| 28.90 | 28.98 | 29.02 | 28.97 | | | | |

(Infrared Absorption Spectrum Measurement)

Infrared absorption spectrum measurement of the crystals obtained in respective Examples was carried out according to the potassium bromide tablet method in the infrared absorption spectrum measurement method as described in the Japanese Pharmacopoeia, General Tests by using FT/1R-620 (JASCO Corporation) with a measurement range of 4000-400 cm⁻¹ and a resolution of 4 cm⁻¹.

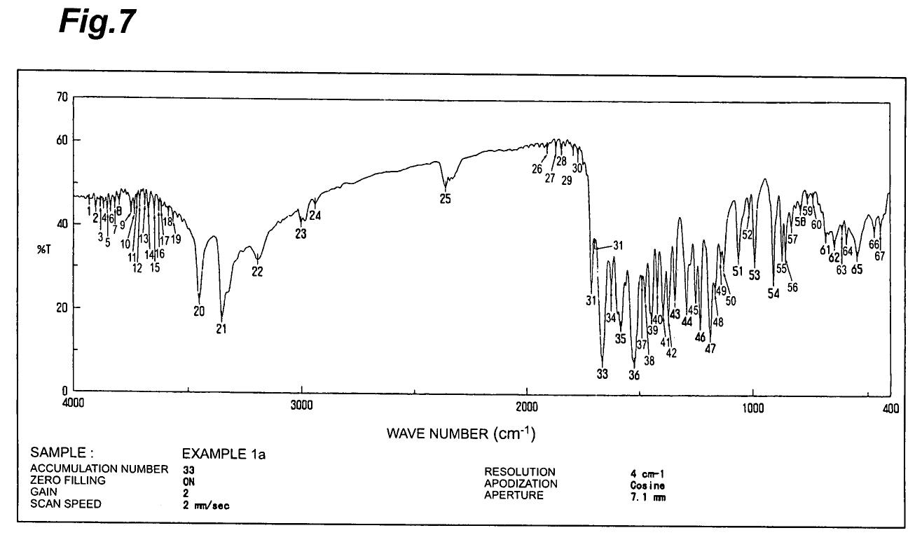

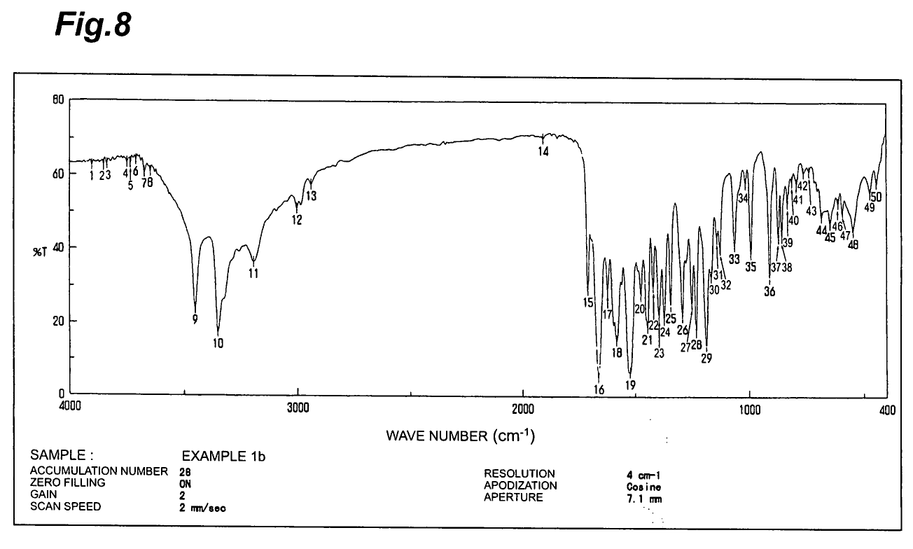

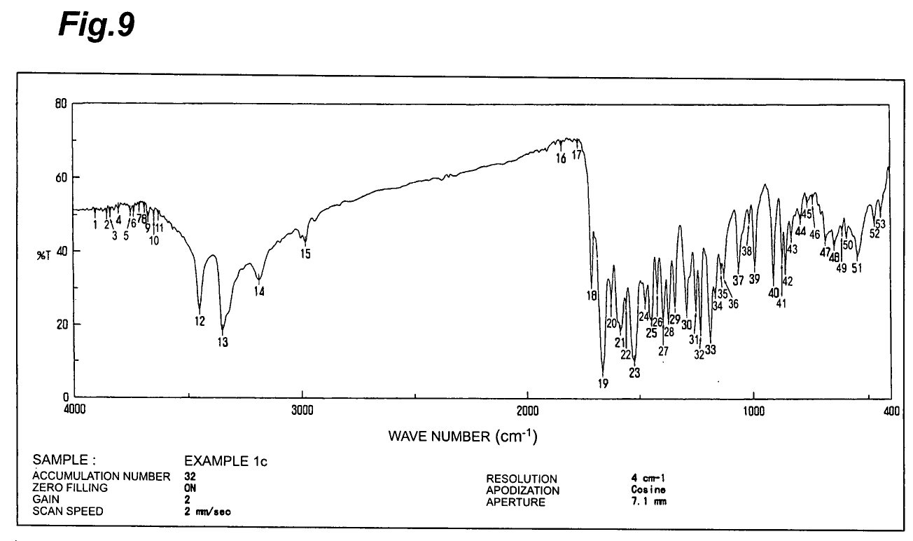

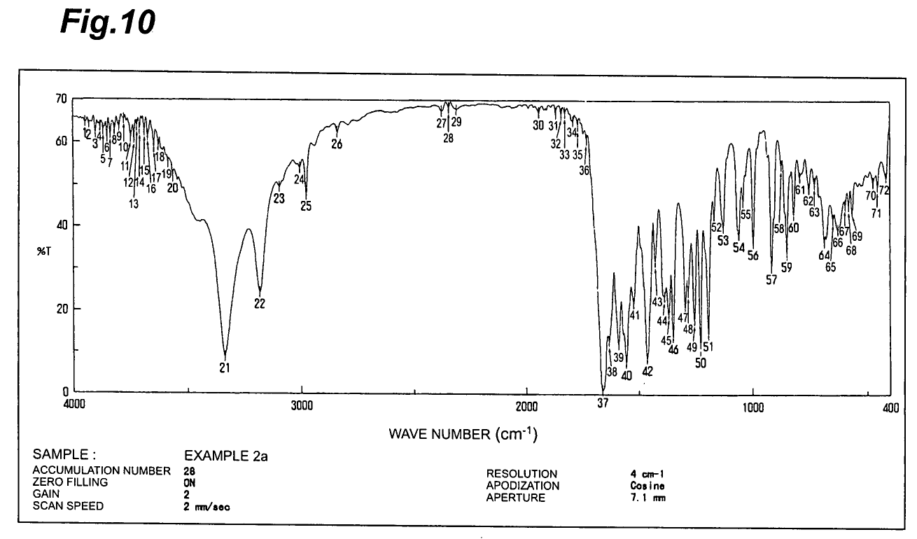

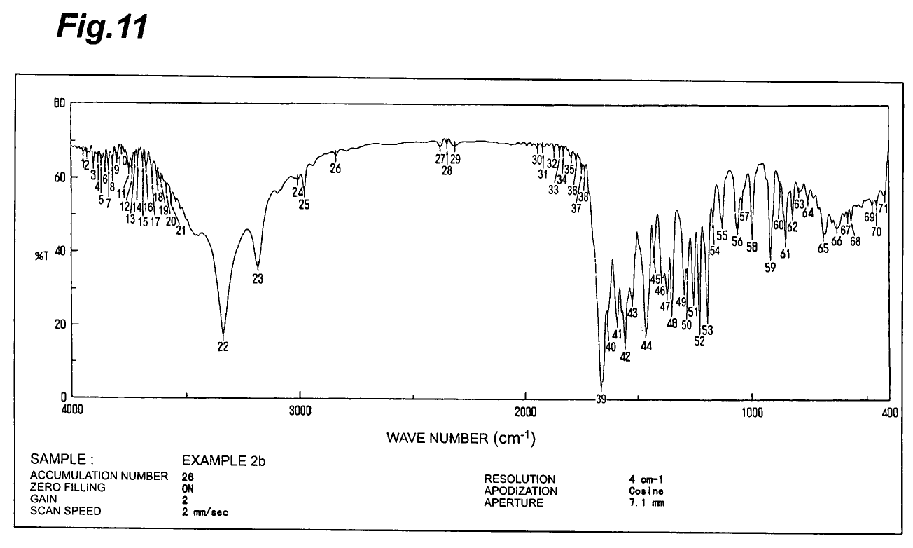

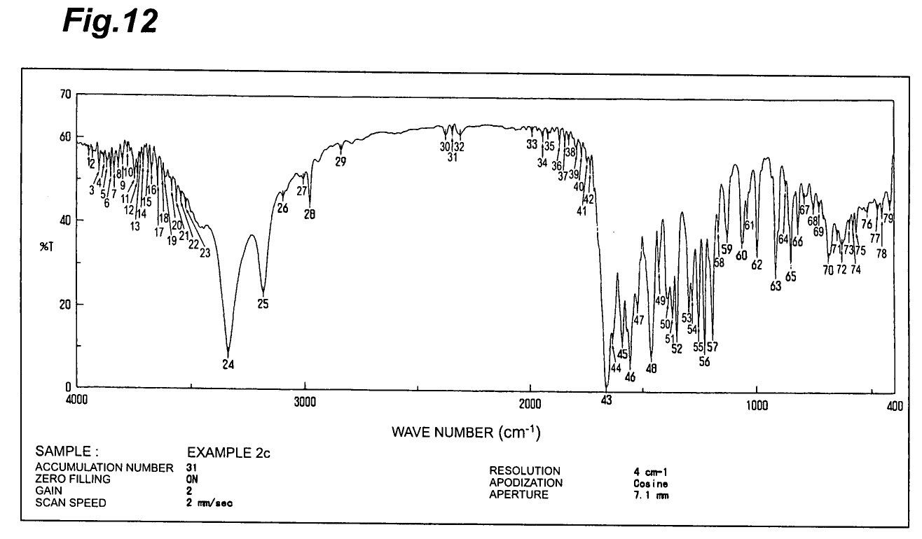

The infrared absorption spectra of the crystals obtained in Examples 1a-1c and 2a-2c are shown in FIG. 7-12, respectively, and wave numbers of the absorption peaks and transmittance (% T) are shown in Tables 8-13, respectively. Further, the peaks of characteristic absorptions in respective Examples and the average values of respective peaks are listed in Table 14.

TABLE 8
SAMPLE: EXAMPLE 1a

| | WAVE | | | WAVE | | | WAVE | | | WAVE | |
|---|---|---|---|---|---|---|---|---|---|---|---|
| PEAK | NUMBER | | PEAK | NUMBER | | PEAK | NUMBER | | PEAK | NUMBER | |
| NUMBER | (cm⁻¹) | % T | NUMBER | (cm⁻¹) | % T | NUMBER | (cm⁻¹) | % T | NUMBER | (cm⁻¹) | % T |
| 1 | 3931.18 | 45.9000 | 2 | 3902.25 | 44.2482 | 3 | 3882.97 | 45.5739 | 4 | 3870.43 | 45.0296 |
| 5 | 3853.08 | 43.8748 | 6 | 3839.58 | 44.4297 | 7 | 3820.29 | 45.3034 | 8 | 3801.01 | 46.0442 |
| 9 | 3749.90 | 44.2226 | 10 | 3735.44 | 43.9472 | 11 | 3723.87 | 46.3443 | 12 | 3711.33 | 46.0532 |
| 13 | 3690.12 | 46.1108 | 14 | 3674.69 | 44.0923 | 15 | 3648.66 | 43.7544 | 16 | 3629.37 | 44.6435 |
| 17 | 3617.80 | 44.4673 | 18 | 3586.95 | 43.2537 | 19 | 3566.70 | 41.4440 | 20 | 3451.96 | 22.3589 |
| 21 | 3352.64 | 18.1744 | 22 | 3195.47 | 31.8660 | 23 | 3003.59 | 40.9804 | 24 | 2941.88 | 45.5361 |
| 25 | 2361.41 | 49.8472 | 26 | 1908.22 | 59.1594 | 27 | 1868.68 | 59.3052 | 28 | 1844.58 | 58.8413 |
| 29 | 1792.51 | 58.7186 | 30 | 1771.30 | 57.8139 | 31 | 1712.48 | 25.6521 | 32 | 1698.02 | 36.3550 |
| 33 | 1664.27 | 8.0307 | 34 | 1624.73 | 26.2601 | 35 | 1583.27 | 16.5974 | 36 | 1523.49 | 7.8357 |
| 37 | 1488.78 | 27.5722 | 38 | 1474.31 | 23.5677 | 39 | 1447.31 | 18.5404 | 40 | 1422.24 | 25.7948 |
| 41 | 1396.21 | 20.3006 | 42 | 1373.07 | 19.2443 | 43 | 1343.18 | 22.5631 | 44 | 1292.07 | 20.8167 |
| 45 | 1251.58 | 23.4114 | 46 | 1232.29 | 17.1507 | 47 | 1186.97 | 14.2968 | 48 | 1164.79 | 25.7644 |
| 49 | 1140.69 | 33.1056 | 50 | 1127.19 | 31.5090 | 51 | 1063.55 | 32.8054 | 52 | 1015.34 | 44.9572 |
| 53 | 992.20 | 31.8739 | 54 | 909.27 | 27.5640 | 55 | 872.63 | 33.5440 | 56 | 857.20 | 34.3007 |
| 57 | 831.17 | 40.4874 | 58 | 790.67 | 44.8201 | 59 | 760.78 | 47.4970 | 60 | 737.64 | 47.7491 |
| 61 | 682.68 | 38.0041 | 62 | 645.07 | 36.1694 | 63 | 611.32 | 39.9503 | 64 | 592.04 | 37.6119 |
| 65 | 544.79 | 33.3718 | 66 | 471.51 | 39.3295 | 67 | 443.55 | 40.0536 | | | |

TABLE 9
SAMPLE: EXAMPLE 1b

| | WAVE | | | WAVE | | | WAVE | | | WAVE | |
|---|---|---|---|---|---|---|---|---|---|---|---|
| PEAK | NUMBER | | PEAK | NUMBER | | PEAK | NUMBER | | PEAK | NUMBER | |
| NUMBER | (cm⁻¹) | % T | NUMBER | (cm⁻¹) | % T | NUMBER | (cm⁻¹) | % T | NUMBER | (cm⁻¹) | % T |
| 1 | 3903.22 | 62.7887 | 2 | 3854.04 | 62.6193 | 3 | 3839.58 | 63.0859 | 4 | 3749.90 | 63.5221 |
| 5 | 3735.44 | 63.3653 | 6 | 3711.33 | 64.2147 | 7 | 3674.69 | 60.8349 | 8 | 3648.66 | 61.1385 |
| 9 | 3452.92 | 23.9773 | 10 | 3352.64 | 17.4978 | 11 | 3196.43 | 36.6064 | 12 | 3004.55 | 51.2164 |
| 13 | 2941.88 | 57.8045 | 14 | 1908.22 | 70.2357 | 15 | 1711.51 | 29.3651 | 16 | 1664.27 | 5.3748 |
| 17 | 1624.73 | 29.4227 | 18 | 1584.24 | 15.5256 | 19 | 1524.45 | 6.6503 | 20 | 1475.28 | 27.7752 |
| 21 | 1447.31 | 19.1425 | 22 | 1422.24 | 30.1585 | 23 | 1398.21 | 22.5288 | 24 | 1374.03 | 21.2650 |
| 25 | 1344.14 | 25.0454 | 26 | 1292.07 | 21.9457 | 27 | 1251.58 | 25.5724 | 28 | 1232.29 | 17.8466 |
| 29 | 1186.97 | 14.1035 | 30 | 1165.76 | 32.8256 | 31 | 1140.69 | 43.4429 | 32 | 1128.15 | 40.7376 |
| 33 | 1064.51 | 41.2862 | 34 | 1015.34 | 58.2909 | 35 | 992.20 | 39.1965 | 36 | 910.24 | 32.5256 |
| 37 | 872.63 | 43.5868 | 38 | 858.17 | 43.7942 | 39 | 832.13 | 53.1289 | 40 | 812.85 | 58.7989 |
| 41 | 791.64 | 58.2784 | 42 | 761.74 | 61.1435 | 43 | 737.64 | 61.4664 | 44 | 683.64 | 49.1766 |
| 45 | 646.04 | 47.2793 | 46 | 611.32 | 52.9664 | 47 | 592.04 | 50.1626 | 48 | 545.76 | 45.2944 |
| 49 | 472.47 | 55.7279 | 50 | 443.55 | 58.3037 | | | | | | |

TABLE 10
SAMPLE: EXAMPLE 1c

| | WAVE | | | WAVE | | | WAVE | | | WAVE | |
|---|---|---|---|---|---|---|---|---|---|---|---|
| PEAK | NUMBER | | PEAK | NUMBER | | PEAK | NUMBER | | PEAK | NUMBER | |
| NUMBER | (cm⁻¹) | % T | NUMBER | (cm⁻¹) | % T | NUMBER | (cm⁻¹) | % T | NUMBER | (cm⁻¹) | % T |
| 1 | 3902.25 | 50.3617 | 2 | 3854.04 | 50.1553 | 3 | 3839.58 | 50.6571 | 4 | 3801.97 | 51.7857 |
| 5 | 3749.90 | 50.9984 | 6 | 3735.44 | 50.8468 | 7 | 3711.33 | 52.1525 | 8 | 3699.16 | 52.0424 |
| 9 | 3673.73 | 49.5143 | 10 | 3648.66 | 49.7618 | 11 | 3629.37 | 50.0407 | 12 | 3451.96 | 24.3465 |
| 13 | 3350.71 | 18.5556 | 14 | 3190.65 | 32.4426 | 15 | 2983.34 | 42.7174 | 16 | 1844.58 | 69.0444 |
| 17 | 1772.26 | 69.6456 | 18 | 1712.48 | 31.6257 | 19 | 1684.27 | 7.4802 | 20 | 1625.70 | 28.1570 |
| 21 | 1585.20 | 18.8340 | 22 | 1560.13 | 25.9825 | 23 | 1523.49 | 10.4464 | 24 | 1474.31 | 26.1905 |
| 25 | 1447.31 | 21.5116 | 26 | 1422.24 | 30.2226 | 27 | 1396.21 | 22.3728 | 28 | 1373.07 | 21.8362 |
| 29 | 1344.14 | 25.2179 | 30 | 1292.07 | 23.7257 | 31 | 1251.58 | 25.5881 | 32 | 1231.33 | 18.8437 |
| 33 | 1186.97 | 16.8881 | 34 | 1164.79 | 28.9811 | 35 | 1139.72 | 35.6581 | 36 | 1127.19 | 34.1104 |
| 37 | 1063.55 | 35.7793 | 38 | 1014.37 | 49.3645 | 39 | 992.20 | 36.2202 | 40 | 909.27 | 32.4686 |
| 41 | 872.63 | 38.5241 | 42 | 857.20 | 36.1720 | 43 | 831.17 | 44.3965 | 44 | 790.67 | 49.3395 |
| 45 | 760.78 | 53.9713 | 46 | 737.64 | 54.8796 | 47 | 683.64 | 43.5492 | 48 | 645.07 | 42.0847 |
| 49 | 610.36 | 46.1061 | 50 | 592.04 | 44.2588 | 51 | 543.83 | 39.1675 | 52 | 471.51 | 48.7501 |
| 53 | 442.58 | 51.2933 | 54 | 403.05 | 62.2511 | | | | | | |

TABLE 11
SAMPLE: EXAMPLE 2a

| | WAVE | | | WAVE | | | WAVE | | | WAVE | |
|---|---|---|---|---|---|---|---|---|---|---|---|
| PEAK | NUMBER | | PEAK | NUMBER | | PEAK | NUMBER | | PEAK | NUMBER | |
| NUMBER | (cm⁻¹) | % T | NUMBER | (cm⁻¹) | % T | NUMBER | (cm⁻¹) | % T | NUMBER | (cm⁻¹) | % T |
| 1 | 3947.57 | 64.6510 | 2 | 3931.18 | 64.2965 | 3 | 3902.25 | 62.1879 | 4 | 3882.00 | 63.6113 |
| 5 | 3870.43 | 62.8534 | 6 | 3853.08 | 61.1682 | 7 | 3839.58 | 61.9789 | 8 | 3820.29 | 62.9568 |
| 9 | 3801.01 | 63.7021 | 10 | 3779.80 | 65.4009 | 11 | 3749.90 | 61.2031 | 12 | 3735.44 | 60.7760 |
| 13 | 3723.87 | 63.7629 | 14 | 3711.33 | 62.9799 | 15 | 3689.18 | 62.7534 | 16 | 3675.86 | 61.5792 |
| 17 | 3648.66 | 58.7798 | 18 | 3629.37 | 59.1510 | 19 | 3586.95 | 55.1412 | 20 | 3565.74 | 51.9090 |
| 21 | 3339.14 | 8.8546 | 22 | 3184.86 | 24.4703 | 23 | 3099.05 | 49.6037 | 24 | 3007.44 | 54.1278 |
| 25 | 2979.48 | 47.8851 | 26 | 2839.67 | 62.5062 | 27 | 2377.80 | 67.4773 | 28 | 2345.98 | 68.3580 |
| 29 | 2311.27 | 67.7863 | 30 | 1943.89 | 67.1878 | 31 | 1868.68 | 67.0682 | 32 | 1844.58 | 66.9751 |
| 33 | 1828.19 | 67.1858 | 34 | 1792.51 | 65.1319 | 35 | 1771.30 | 64.4117 | 36 | 1732.73 | 60.9836 |
| 37 | 1662.34 | 0.9623 | 38 | 1634.38 | 12.8838 | 39 | 1591.95 | 12.0549 | 40 | 1558.20 | 7.5272 |
| 41 | 1524.45 | 22.1174 | 42 | 1464.67 | 8.5881 | 43 | 1429.96 | 32.0119 | 44 | 1388.50 | 23.6552 |
| 45 | 1370.18 | 19.5405 | 46 | 1350.89 | 13.8898 | 47 | 1296.89 | 21.5407 | 48 | 1281.47 | 24.8695 |
| 49 | 1255.43 | 18.0553 | 50 | 1228.43 | 10.5935 | 51 | 1193.72 | 14.5053 | 52 | 1167.69 | 43.1354 |
| 53 | 1127.19 | 40.0860 | 54 | 1060.66 | 38.5032 | 55 | 1042.34 | 46.3845 | 56 | 997.02 | 36.5950 |
| 57 | 916.02 | 30.8092 | 58 | 874.56 | 55.0132 | 59 | 850.45 | 33.7215 | 60 | 819.60 | 43.2136 |
| 61 | 792.60 | 52.1763 | 62 | 752.10 | 49.4830 | 63 | 728.00 | 50.7867 | 64 | 686.53 | 38.6977 |
| 65 | 647.96 | 42.1516 | 66 | 626.75 | 39.6482 | 67 | 594.93 | 45.6731 | 68 | 579.50 | 45.4091 |
| 69 | 565.04 | 42.9857 | 70 | 474.40 | 51.0301 | 71 | 455.12 | 50.0223 | 72 | 417.51 | 52.0934 |

TABLE 12
SAMPLE: EXAMPLE 2b

| | WAVE | | | WAVE | | | WAVE | | | WAVE | |
|---|---|---|---|---|---|---|---|---|---|---|---|
| PEAK | NUMBER | | PEAK | NUMBER | | PEAK | NUMBER | | PEAK | NUMBER | |
| NUMBER | (cm⁻¹) | % T | NUMBER | (cm⁻¹) | % T | NUMBER | (cm⁻¹) | % T | NUMBER | (cm⁻¹) | % T |
| 1 | 3947.57 | 66.9343 | 2 | 3931.18 | 66.5212 | 3 | 3902.25 | 63.8862 | 4 | 3882.00 | 65.5681 |
| 5 | 3870.43 | 64.6510 | 6 | 3853.08 | 62.8065 | 7 | 3838.61 | 63.5944 | 8 | 3820.29 | 64.5792 |
| 9 | 3801.01 | 85.3683 | 10 | 3780.76 | 67.5582 | 11 | 3749.90 | 62.4268 | 12 | 3735.44 | 62.1397 |
| 13 | 3723.87 | 65.4550 | 14 | 3711.33 | 64.5822 | 15 | 3689.16 | 64.4015 | 16 | 3674.89 | 63.0108 |
| 17 | 3648.66 | 59.9574 | 18 | 3628.41 | 60.6478 | 19 | 3610.09 | 59.7570 | 20 | 3586.95 | 57.2073 |
| 21 | 3565.74 | 54.0188 | 22 | 3339.14 | 17.3207 | 23 | 3185.83 | 35.9208 | 24 | 3008.41 | 59.6548 |
| 25 | 2979.48 | 56.3115 | 26 | 2839.67 | 66.1140 | 27 | 2376.84 | 68.7358 | 28 | 2345.98 | 69.5194 |
| 29 | 2310.30 | 68.8212 | 30 | 1942.93 | 68.4156 | 31 | 1920.75 | 68.6540 | 32 | 1868.68 | 67.5680 |
| 33 | 1844.58 | 67.4810 | 34 | 1828.19 | 67.7038 | 35 | 1792.51 | 65.9869 | 36 | 1771.30 | 65.1128 |
| 37 | 1748.16 | 63.1139 | 38 | 1732.73 | 62.3721 | 39 | 1662.34 | 3.5651 | 40 | 1835.34 | 23.1958 |
| 41 | 1591.95 | 21.1624 | 42 | 1557.24 | 15.0986 | 43 | 1524.45 | 27.1589 | 44 | 1464.67 | 18.1794 |
| 45 | 1428.99 | 40.2445 | 46 | 1395.25 | 33.3128 | 47 | 1371.14 | 28.8236 | 48 | 1349.93 | 24.3173 |
| 49 | 1295.93 | 30.3197 | 50 | 1281.47 | 34.4593 | 51 | 1255.43 | 27.4197 | 52 | 1229.40 | 19.3922 |
| 53 | 1193.72 | 22.4587 | 54 | 1167.69 | 49.9615 | 55 | 1127.19 | 48.2969 | 56 | 1061.62 | 46.7331 |
| 57 | 1042.34 | 53.3130 | 58 | 997.02 | 45.1946 | 59 | 916.02 | 39.5083 | 60 | 874.56 | 58.2522 |
| 61 | 851.42 | 43.2948 | 62 | 819.80 | 50.6987 | 63 | 792.60 | 56.7426 | 64 | 752.10 | 54.6364 |
| 65 | 686.53 | 44.8873 | 66 | 627.72 | 46.8548 | 67 | 579.50 | 49.8957 | 68 | 565.04 | 48.7841 |
| 69 | 474.40 | 53.2674 | 70 | 455.12 | 53.3351 | 71 | 418.48 | 55.7359 | | | |

TABLE 13
SAMPLE: EXAMPLE 2c

| | WAVE | | | WAVE | | | WAVE | | | WAVE | |
|---|---|---|---|---|---|---|---|---|---|---|---|
| PEAK | NUMBER | | PEAK | NUMBER | | PEAK | NUMBER | | PEAK | NUMBER | |
| NUMBER | (cm⁻¹) | % T | NUMBER | (cm⁻¹) | % T | NUMBER | (cm⁻¹) | % T | NUMBER | (cm⁻¹) | % T |
| 1 | 3947.57 | 56.7484 | 2 | 3931.18 | 56.2460 | 3 | 3902.25, | 53.0225 | 4 | 3882.00 | 55.0310 |
| 5 | 3870.43 | 53.9317 | 6 | 3853.08 | 51.7187 | 7 | 3838.61 | 52.6328 | 8 | 3820.29 | 53.8496 |
| 9 | 3801.01 | 54.8032 | 10 | 3780.76 | 57.6637 | 11 | 3748.94 | 51.2303 | 12 | 3735.44 | 50.9972 |
| 13 | 3723.87 | 55.0212 | 14 | 3711.33 | 54.1190 | 15 | 3689.16 | 53.9965 | 16 | 3674.69 | 52.5972 |
| 17 | 3648.66 | 49.5453 | 18 | 3628.41 | 51.0640 | 19 | 3616.84 | 50.4203 | 20 | 3586.95 | 48.5140 |
| 21 | 3565.74 | 45.5294 | 22 | 3545.49 | 45.9023 | 23 | 3524.27 | 44.3791 | 24 | 3339.14 | 8.6318 |
| 25 | 3184.86 | 23.5096 | 26 | 3099.05 | 46.2331 | 27 | 3007.44 | 50.4299 | 28 | 2979.48 | 44.8418 |
| 29 | 2839.67 | 57.4731 | 30 | 2376.84 | 61.3115 | 31 | 2345.98 | 62.1115 | 32 | 2310.30 | 61.2994 |
| 33 | 1991.14 | 62.1221 | 34 | 1942.93 | 61.1286 | 35 | 1920.75 | 61.6056 | 36 | 1868.68 | 60.1388 |
| 37 | 1844.58 | 60.0459 | 38 | 1828.19 | 60.3761 | 39 | 1792.51 | 58.1187 | 40 | 1771.30 | 57.1056 |
| 41 | 1748.16 | 54.6730 | 42 | 1732.73 | 53.9627 | 43 | 1662.34 | 1.0762 | 44 | 1635.34 | 12.8451 |
| 45 | 1591.95 | 12.0388 | 46 | 1557.24 | 6.5300 | 47 | 1523.49 | 20.4790 | 48 | 1463.71 | 8.5892 |
| 49 | 1429.96 | 30.2743 | 50 | 1388.50 | 22.7410 | 51 | 1370.18 | 19.2561 | 52 | 1349.93 | 13.4266 |
| 53 | 1296.89 | 20.8356 | 54 | 1281.47 | 23.4625 | 55 | 1255.43 | 17.1433 | 56 | 1226.43 | 10.3828 |
| 57 | 1193.72 | 13.9089 | 58 | 1167.69 | 39.8446 | 59 | 1128.15 | 37.4359 | 60 | 1064.51 | 35.9921 |
| 61 | 1042.34 | 42.9687 | 62 | 997.02 | 33.7870 | 63 | 916.02 | 28.8189 | 64 | 874.56 | 49.6391 |
| 65 | 850.45 | 31.1042 | 66 | 819.60 | 39.4562 | 67 | 792.60 | 46.6446 | 68 | 752.10 | 44.1803 |
| 69 | 728.00 | 44.6814 | 70 | 686.53 | 32.5322 | 71 | 648.93 | 38.0168 | 72 | 627.72 | 35.8481 |
| 73 | 594.93 | 40.6210 | 74 | 579.50 | 40.0418 | 75 | 565.04 | 38.4537 | 76 | 518.76 | 43.5653 |
| 77 | 474.40 | 44.1801 | 78 | 455.12 | 43.1782 | 79 | 420.41 | 45.1423 | | | |

TABLE 14

| Polymorph (A), wave number (cm⁻¹) | | | | Polymorph (B), wave number (cm⁻¹) | | | |
|---|---|---|---|---|---|---|---|
| Ex. 1a | Ex. 1b | Ex. 1c | Ave. | Ex. 2a | Ex. 2b | Ex. 2c | Ave. |
| 3451.96 | 3452.92 | 3451.96 | 3452.28 | 1558.20 | 1557.24 | 1557.24 | 1557.56 |
| 1712.48 | 1711.51 | 1712.48 | 1712.16 | 1464.67 | 1464.67 | 1463.71 | 1464.35 |

(Purity Test of the Polymorph (A))

In Example 1a, the purities of 4-(3-chloro-4-(cyclopropylaminocarbonyl)aminophenoxy)-7-methoxy-6-quinolinecarboxamide anterior and posterior to crystallization were measured according to the following method.

In Example 1a, a portion of the reaction mixture after being heated and stirred at 55° C. for 20 hours and further at 60° C. for 4 hours was collected, and it was subjected to HPLC as a sample anterior to crystallization. On the other hand, the polymorph (A) obtained in Example 1a was subjected to HPLC as a sample posterior to crystallization.

The conditions of HPLC were as follows.

Column: ODS column (Mightysil RP-18 GP, Kanto Kagaku KK; inner diameter 4.6 mm, column length 150 mm, particle size 3 μm)

Column temperature: 40° C. (using a column oven)

Mobile phase:

Solution A H₂O:CH₃CN:HClO₄*=990:10:1 (v/v/v)

Solution B H₂O:CH₃CN:HClO₄*=100:900:1 (v/v/v)

(*: 70% aqueous solution)

Eluted by the linear gradient shown in Table 15

TABLE 15

| time (minute) | B conc. (%) |
|---|---|
| 0 | 5 |
| 3 | 20 |
| 15 | 20 |
| 30 | 100 |

Flow rate: 1.0 mL/min
Detection: UV detector (wavelength: 252 nm)

The contents (the ratio of peak areas) of 4-(3-chloro-4-(cyclopropylaminocarbonyl)aminophenoxy)-7-methoxy-6-quinolinecarboxamides and impurities in the samples anterior and posterior to crystallization to the polymorph (A) are shown in Table 16.

TABLE 16

| substance | P | Q | R |
|---|---|---|---|
| anterior | 1.26 | 3.65 | 92.4 |
| posterior | 0.49 | not | 97.6 |

In Tables 16 and 17, P represents 7-methoxy-4-chloro-quinoline-6-carboxamide, Q represents 1-(2-chloro-4-hydroxyphenyl)-3-cyclopropylurea, and R represents 4-(3-chloro-4-(cyclopropylaminocarbonyl)aminophenoxy)-7-methoxy-6-quinolinecarboxamide.

4-(3-chloro-4-(cyclopropylaminocarbonyl)aminophenoxy)-7-methoxy-6-quinolinecarboxamide was 92.4% in purity anterior to crystallization, but 97.6% in purity posterior to crystallization to the polymorph (A), indicating that the crystallization improved the purity.

(Purity Test of the Polymorph (B))

In Example 2a, the purities of 4-(3-chloro-4-(cyclopropylaminocarbonyl)aminophenoxy)-7-methoxy-6-quinolinecarboxamide anterior and posterior to crystallization were measured according to the following method.

In Example 2a, a portion of the reaction mixture after being heated and stirred at 60° C. for 25 hours was collected, and it was subjected to HPLC as a sample anterior to crystallization. On the other hand, the polymorph (B) obtained in Example 2a was subjected to HPLC as a sample posterior to crystallization. The conditions of HPLC were the same as those above-described in the purity test for the polymorph (A).

The contents (the ratio of peak areas) of 4-(3-chloro-4-(cyclopropylaminocarbonyl)aminophenoxy)-7-methoxy-6-quinolinecarboxamides and impurities in the samples anterior and posterior to crystallization to the polymorph (B) are shown in Table 17.

TABLE 17

| substance | P | Q | R |
|---|---|---|---|
| anterior | 0.46 | 3.48 | 92.2 |
| posterior | 0.05 | not | 98.1 |

4-(3-chloro-4-(cyclopropylaminocarbonyl)aminophenoxy)-7-methoxy-6-quinolinecarboxamide was 92.2% in purity anterior to crystallization, but 98.1% in purity posterior to crystallization to the polymorph (B), indicating that the crystallization improved the purity. Also, the purity was higher compared with that of the polymorph (A), that is, 97.6%. This revealed that the crystallization operation to the polymorph (B) was superior to that to the polymorph (A) in the efficiency of removing the impurities.

(Hygroscopicity Test by a Desiccator Method)

The hygroscopicities of the crystals obtained in Examples 1d and 2d were evaluated by a desiccator method. The crystals were stored for 1 week under the conditions as shown in Table 18, and then appearance observation, powder X-ray diffraction measurement, and water content measurement were carried out. Weighing bottles (in opened caps) were used for containers, and MIR-552 (Sanyo) was used for a storage apparatus.

TABLE 18

| condition | temperature | relative humidity | desiccator |
|---|---|---|---|
| A | 25° C. | 75% | NaCl |
| | | | saturated |
| B | 25° C. | 93% | KNO₃ |
| | | | saturated |

The powder X-ray diffraction analysis was carried out under the following conditions.

Apparatus: RINT2000 manufactured by Rigaku Denki KK

Sample holder: glass holder (diameter 10 mm)

Target: Cu

Detector: Scintillation counter

Tube voltage: 40 kV

Tube current: 200 mA

Slit: DS ½°, RS 0.3 mm, SS ½°

Scan speed: 2°/min

Step/sampling: 0.02°

Scan range: 5-40°

Goniometer: Vertical goniometer

Filter: not used

The water content was measured (by the Karl Fischer method) by using the following apparatus and reagents.

Apparatus: Moisture meter CA-06 (Mitsubishi Chemical)

Reagents:

Lactose monohydrate NF (Mallinckrodt)

Karl Fischer reagents:

Anode solution/Aquamicron AX (Mitsubishi Chemical)

Cathode solution/Aquamicron CXU (Mitsubishi Chemical)

The results of evaluating the hygroscopicities of the crystals obtained in Examples 1d and 2d are listed in Tables 19 and 20, respectively.

TABLE 19

| | | water content | powder X-ray |
|---|---|---|---|
| condition | appearance | (wt %) | diffraction pattern |
| prior to storage | light brown | 1.0 | A |
| | powder | | |
| A | light brown | 1.0 | A |
| | powder | | |
| B | light brown | 1.2 | A |
| | powder | | |

TABLE 20

| | | water content | powder X-ray |
|---|---|---|---|
| condition | appearance | (wt %) | diffraction pattern |
| prior to storage | pale brownish | 0.5 | B |
| | white powder | | |
| A | pale brownish | 0.5 | B |
| | white powder | | |
| B | pale brownish | 0.5 | B |
| | white powder | | |

As is evident from the results shown in Tables 19 and 20, both of the crystals obtained in Examples 1d and 2d had no perceivable hygroscopicitiy and no perceivable crystal transition.

(Hygroscopicity Test by Microbalance Method)

The higroscopicities of the crystals obtained in Examples 1d and 2d were evaluated by microbalance method. An apparatus and conditions employed were as follows.

Apparatus: Integrated microbalance system MB 300W (VTI Co.)

Temperature: 25° C.

Relative humidity step: 5 to 95 by 5

Equilibrium Criteria: 0.0050 wt % (5 minutes)

Maximum equilibrium time: 120 minutes

Initial dry: on

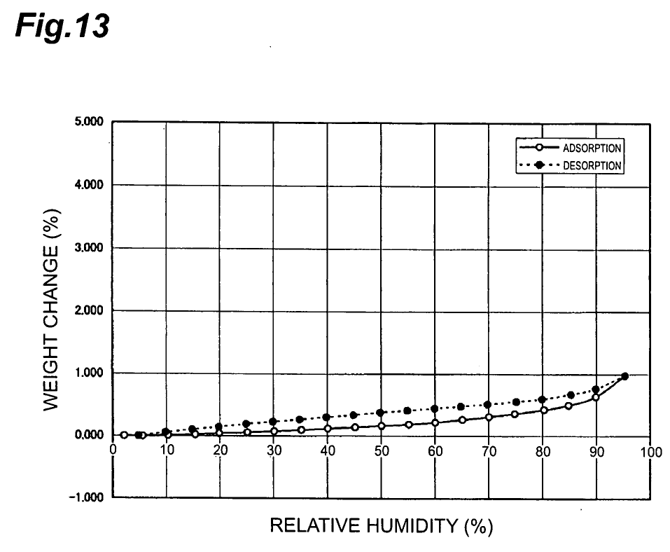

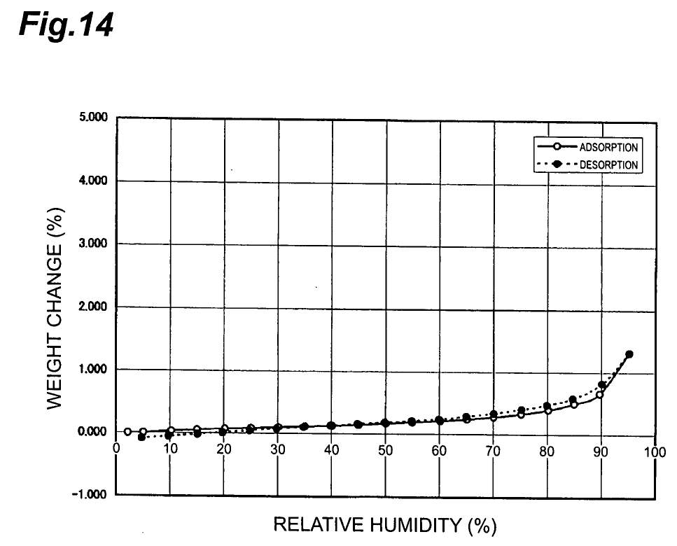

The results of measuring the higroscopicities of the crystals obtained in Examples 1d and 2d by microbalance method are shown in FIGS. 13 and 14, respectively. As is seen from the results shown in these figures, within the range of 5-95% of relative humidity, the polymorph (A) gave a weight change of 1% and the polymorph (B) gave that of 1.5%. Both of the polymorphs, therefore, had no perceivable hygroscopisity.

(Solid Stability Test)

The solid stabilities of the crystal obtained in Examples 1d and 2d were evaluated. The crystals were stored for 1 month under the conditions as shown in Table 21, and then appearance observation, water content measurement (by the Karl Fischer method), purity test and residual ratio (percent) measurement by HPLC, and powder X-ray diffraction measurement were carried out. The water content measurement and the powder X-ray diffraction measurement were carried out by the same method as described in the hygroscopicity test by the dessiccator method. Further, the purity test and the residual ratio (percent) measurement by HPLC were carried out by the same method as described above, except for the condition that the column temperature was 35° C. In this connection, the residual ratio (percent) (measurement by HPLC) was defined as stated bellow by using the crystal stored under the condition C as the standard and its solution as the standard solution.
Remaining percent (%)=[(Peak area of the sample solution)×(Weighed amount of the standard: in terms of a dehydrate (mg))]×100/[(Peak area of the standard solution)×(Weighed amount of the sample: in terms of a dehydrate (mg))]

TABLE 21

| | temperature | | | storage |
|---|---|---|---|---|
| condition | etc. | container | cap | apparatus |
| C | −20° C. | brown screw vial | closed | PU-1F*¹ |
| D | 25° C., 1000lx | shading with | closed | LT-120*² |
| | | aluminum foil, | | |
| | | quartz tube | | |
| E | 25° C., 1000lx | quartz tube | closed | LT-120*² |
| F | 40° C., 75% RH | brown screw vial | open | LH21- |
| G | 60° C. | brown screw vial | closed | DN-61*³ |

*¹Tabai Espec KK
*²Nagano Science KK
*³Yamato Science KK

The results of evaluating solid stabilities of the crystals obtained in Examples 1d and 2d are listed in Tables 22 and 23, respectively.

TABLE 22

| | | | | | powder |
|---|---|---|---|---|---|
| | | water | | remaining | X-ray |
| | | content | impurity | percent | diffraction |
| condition | appearance | (wt %) | (%) | (%) | pattern |
| prior to | light brown | 1.0 | 2.71 | — | A |
| storage | powder | | | | |
| C | light brown | 1.0 | 2.66 | (100) | A |
| | powder | | | | |
| D | light brown | 0.7 | 2.67 | 103.3 | A |
| | powder | | | | |
| E | light brown | 0.8 | 2.68 | 104.3 | A |
| | powder | | | | |
| F | light brown | 1.2 | 2.65 | 102.3 | A |
| | powder | | | | |
| G | light brown | 0.5 | 2.65 | 104.4 | A |
| | powder | | | | |

TABLE 23

| | | | | | powder |
|---|---|---|---|---|---|
| | | water | | remaining | X-ray |
| | | content | impurity | percent | diffraction |
| condition | appearance | (wt %) | (%) | (%) | pattern |
| prior to | pale brownish | 0.5 | 1.53 | — | B |
| storage | white powder | | | | |
| C | pale brownish | 0.4 | 1.55 | (100) | B |
| | white powder | | | | |
| D | pale brownish | 0.3 | 1.54 | 101.8 | B |
| | white powder | | | | |
| E | pale brownish | 0.3 | 1.55 | 100.5 | B |
| | white powder | | | | |
| F | pale brownish | 0.4 | 1.54 | 100.4 | B |
| | white powder | | | | |
| G | pale brownish | 0.5 | 1.53 | 101.3 | B |
| | white powder | | | | |

As is evident from the results shown in Tables 22-23, no change was observed in the polymorphs (A) and (B) under any storage conditions.

(Solubility Test)

The solubilities (pH 3) of the crystals obtained in Examples 1d and 2d were evaluated by the following method. About 3 mg of the crystals obtained in Examples 1d and 2d were weighed and each of them was put in a 10 mL screw-capped transparent test tube. 5 mL of a buffer solution (Britton Robinson buffer, pH 3.091, ionic strength I=0.3) was added to each of the test tubes to prepare the test solutions.

The test tubes were wrapped with aluminum foil to shield from light, and shaken by a shaker (MS-1 Iuchi Seieido) in the following conditions.

Temperature: 25-26° C. (a temperature in a laboratory)

Shaking frequency: 150 times/minute

Shaking time: 3 hours and 5 hours

Respective sample solutions after shaking were filtered (0.2 μM, Sample LCR13-LG, Millipore Co.), and each 1 mL of the initial filtrate was discarded. Each of accurately pipetted 1 mL of the filtrates was put in a 10 mL test tube, to which accurately pipetted 1 mL of a mixed solution of water/acetonitrile (1:1 (v/v)) was added to prepare a solution for the HPLC analysis.

The HPLC conditions were as follows.

Column: ODS column (Mightysil RP-18GP; inner diameter 4.6 mm, column length 150 mm, particle size 3 μm, manufactured by Kanto Kagaku KK)

Column temperature: 35° C.

Mobile phase:

Solution A H₂O:CH₃CN:HClO₄*=990:10:1 (v/v/v)

Solution B H₂O:CH₃CN:HClO₄*=100:900:1 (v/v/v)

(*: 70% aqueous solution)

Isocratic elution by B=20%

Flow rate: 1.0 mL/min

Detection: UV detector (wavelength: 252 nm)

A standard solution for the HPLC analysis were prepared as follows. About 10 mg of the crystals obtained in Example 2d was accurately weighed, to which a mixed solution of water/acetonitrile/ammonium acetate (100:100:0.1, v/v/w) was added to give accurate 100 mL to prepare a stock standard solution. Accurately pipetted 5 mL of the stock control solution was added with a mixed solution of water/acetonitrile/ammonium acetate (100:100:0.1, v/v/w) to give accurate 25 mL to prepare the standard solution for the HPLC analysis. Regarding a blank solution, a mixed solution of water/acetonitrile/ammonium acetate (100:100:0.1, v/v/w) was used.

The standard solution and respective filtrates were analyzed by HPLC to measure concentrations (mg/mL) of 4-(3-chloro-4-(cyclopropylaminocarbonyl)aminophenoxy)-7-methoxy-6-quinolinecarboxamide in respective filtrates according to the following equation.
Concentration (mg/mL)=(Concentration in the standard solution, mg/mL)×[(Peak area in each filtrate)×2/(Peak area in the standard solution)]

The respective results of the solubility test for the crystals obtained in Examples 1d and 2d are listed in Table 24. The pH of the respective filtrates are listed in Table 25. As is evident from the results, there was no significant difference in the solubility at pH 3 between the polymorphs (A) and (B).

TABLE 24

| shaking time | Example 1d | Example 2d |
|---|---|---|
| 3 hours | 7.7 × 10⁻² | 6.2 × 10⁻² |
| 5 hours | 7.1 × 10⁻² | 5.4 × 10⁻² |

(mg/mL)

TABLE 25

| shaking time | Example 1d | Example 2d |
|---|---|---|
| 3 hours | 3.123 | 3.109 |
| 5 hours | 3.107 | 3.106 |

c-Kit kinase inhibition by 4-(3-Chloro-4-(cyclopropylaminocarbonyl)aminophenoxy)-7-methoxy-6-quinolinecarboxamide was tested in the following Test Example 1 to 4.

### Test Example 1

### Effect on Cell Proliferation Stimulated by SCF

4-(3-Chloro-4-(cyclopropylaminocarbonyl)aminophenoxy)-7-methoxy-6-quinolinecarboxamide was tested for their effects on the proliferation of the small cell lung cancer cell line H-526 expressing c-Kit kinase (purchased from ATCC: CRL-5811).

4-(3-Chloro-4-(cyclopropylaminocarbonyl)aminophenoxy)-7-methoxy-6-quinolinecarboxamide was prepared similarly to the method described in Preparation Examples 1 to 3.

H-526 cells were cultured in a 5% CO₂ incubator (37° c.) using an RPMI1640 medium (Nissui Pharmaceutical Co., Ltd.) containing 10% FCS (purchased from Cell Culture Technologies). After culturing, H-526 cells were washed with PBS three times and were suspended in an RPMI1640 medium containing 0.1% BSA (Sigma Corporation) (hereinafter abbreviated as “BSA-RPMI1640”) at 1.0×10⁵ cells/ml. Each 50 μl of this cell suspension was inoculated to each well of a round bottom 96-well plate, and the suspension was cultured in a 5% CO₂ incubator (37° c.) overnight. After culturing overnight, 50 μl of BSA-RPMI1640 containing 200 ng/ml SCF (R&D Co., Ltd.) and 100 μl of BSA-RPMI1640 containing a diluted test substance (4-(3-Chloro-4-(cyclopropylaminocarbonyl)aminophenoxy)-7-methoxy-6-quinolinecarboxamide) were added to each well.

On the 7th day after addition of the test substance, 20 μl of Cell Counting Kit-8 (Dojin Laboratories) was added to the well and was cultured in a 5% CO₂ incubator (37° c.) for about 2 hours. After color development, the absorbance of each well was determined using a MTP-32 plate reader (Colona Electric Co., Ltd.) at a measuring wavelength of 450 nm and at a reference wavelength of 660 nm. The absorbance of each well was subtracted by the absorbance of the well without addition of SCF, and then the ratio of the absorbance of the well with addition of the test substance to the ratio of the absorbance of the well without addition of the test substance was determined. This ratio was used to calculate the concentration of the test substance required for 50% inhibition of the cell proliferation (IC₅₀).

Consequently, IC₅₀ of 4-(3-Chloro-4-(cyclopropylaminocarbonyl)aminophenoxy)-7-methoxy-6-quinolinecarboxamide was 9.46 nM. The compound inhibited the cell proliferation stimulated by SCF, and was considered to possess c-Kit kinase inhibitory activity. The IC₅₀ of the compound KRN633, which is described in Kazuo Kubo et al., 22nd Symposium on Medicinal Chemistry, Abstracts, pp. 275-277, 2P-320, 2002, proved to be 301 nM and the compound showed only weak activity as compared to 4-(3-Chloro-4-(cyclopropylaminocarbonyl)aminophenoxy)-7-methoxy-6-quinolinecarboxamide. STI571 known as a c-Kit kinase inhibitor showed IC₅₀ of 190 nM.

### Example 2

### Effect on c-Kit Kinase Phosphorylation by SCF Stimulation)

4-(3-Chloro-4-(cyclopropylaminocarbonyl)aminophenoxy)-7-methoxy-6-quinolinecarboxamide was tested for its effect on the phosphorylation of the c-Kit kinase molecule by SCF stimulation in the small cell lung cancer cell line H-526 expressing c-Kit kinase.

H-526 cells were cultured in a 5% CO₂ incubator (37° c.) using an RPMI1640 medium containing 10% FCS. After culturing, H-526 cells were washed with PBS three times and were suspended in a BSA-RPMI1640 medium at 5.0×10⁵ cells/ml. Each 1 ml of this cell suspension was inoculated to the well of a 24-well plate and the suspension was cultured in a 5% CO₂ incubator (37° c.) for 6 hours. After 6-hours culturing, 1 ml of BSA-RPMI1640 containing a diluted test substance (4-(3-Chloro-4-(cyclopropylaminocarbonyl)aminophenoxy)-7-methoxy-6-quinolinecarboxamide) was added to each well and culturing was carried out in a 5% CO₂ incubator (37° c.) for 1 hour. Additional culturing was then carried out in a 5% CO₂ incubator (37° c.) for 5 minutes after the addition of 10 μl of SCF (10 μg/ml, R&D Corporation). After 5-minutes culturing, the cells were washed with PBS and 100 μl of SDS sample loading buffer was added to the cells to prepare a cell lysate sample. After the sample was heat-treated at 94° c. for 10 minutes, it was cryopreserved at −20° c.

The cell lysate sample, 20 μl, was then electrophoresed on a 4-20% gradient polyacrylamide gel (Daiichi Pure Chemicals Co., Ltd.). After electrophoresis, the sample was transferred to a PVDF membrane (Amersham Pharmacia Biotech Inc.) for 3 hours. The transferred membrane was subjected to immunoblot using a phospho-c-kit (Tyr719) antibody (Cell Signaling Technology Inc.) as a primary antibody and an anti-rabbit IgG, HRP-linked antibody (Cell Signaling Technology Inc.) as a secondary antibody. After the membrane was washed, it was developed with a Super Signal (Pierce Biotechnology, Inc.).

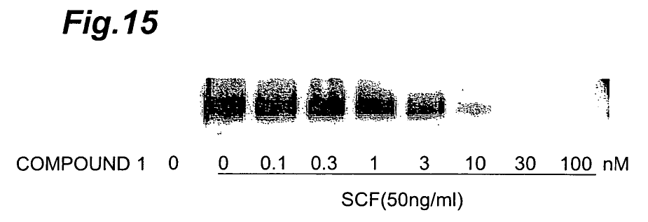

As the results are shown in FIG. 15, c-Kit kinase was not phosphorylated (the farthest left lane) in the absence of SCF, and the addition of 4-(3-chloro-4-(cyclopropylaminocarbonyl)aminophenoxy)-7-methoxy-6-quinolinecarboxamide (“compound 1” in figures) suppressed the c-Kit kinase phosphorylation that would take place in the presence of SCF in a concentration-dependent manner. The phosphorylation inhibitory activity of STI571, which is known as a c-Kit kinase inhibitor, was approximately one tenth of that of 4-(3-chloro-4-(cyclopropylaminocarbonyl)aminophenoxy)-7-methoxy-6-quinolinecarboxamide.

### Example 3

### Effect on Growth of H-526 Tumor Transplanted to Nude Mice

H-526 cells were cultured in a 5% CO₂ incubator (37° c.) using an RPMI1640 medium containing 10% FCS. After the culture medium was collected, H-526 cells were washed with PBS twice and were suspended in PBS at 5.0×10⁷ cells/ml. This cell suspension (0.1 ml) was transplanted to the subcutaneous parts of the right flank of 6-week female Balb/c nu/nu mice (purchased from Charles River Laboratories, Inc.). After transplantation, administration of a test substance (4-(3-chloro-4-(cyclopropylaminocarbonyl)aminophenoxy)-7-methoxy-6-quinolinecarboxamide) was started at the point the tumor volume reached approximately 150 mm³, and thus, oral administration was conducted twice daily for a period of 14 days. The test substance was suspended in a 0.5% methylcellulose solution (Wako Pure Chemical Industries Co., Ltd.) so as to give a dose of 0.1 ml/10 g body weight.

The tumor volume was measured with a caliper twice weekly during the administration period. The long and short diameters of the tumor were measured with a caliper and the tumor volume was calculated according to the equation: ½×long diameter×short diameter×short diameter. Here, the experiment was conducted in a vehicle control group of 10 animals (solvent-administered group) as well as in a test substance administered group of 5 animals.

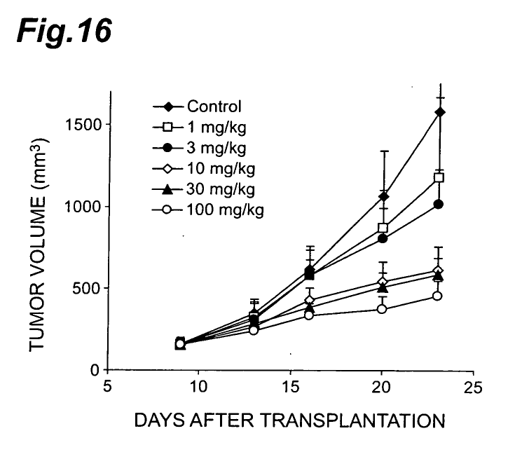

As the results are shown in FIG. 16, 4-(3-chloro-4-(cyclopropylaminocarbonyl)aminophenoxy)-7-methoxy-6-quinolinecarboxamide suppressed the growth of the nude mouse transplanted H-526 tumor in a dose-dependent manner. On the other hand, STI571 known as a c-Kit kinase inhibitor showed little anti-tumor effect when administered even at 160 mg/kg.

### Example 4

### Effect on c-Kit Kinase Phosphorylation in H-526 Tumor Transplanted to Nude Mice

0.1 ml of a H-526 cell suspension prepared at a concentration of 5.0×10⁷ cells/ml, was transplanted to the subcutaneous parts of the right latus of 6-week female Balb/c nu/nu mice (purchased from Charles River Laboratories, Inc.). The animals were then divided into a vehicle control group (solvent-administered group) and a test substance (4-(3-chloro-4-(cyclopropylaminocarbonyl)aminophenoxy)-7-methoxy-6-quinolinecarboxamide) administered group at the point the tumor volume reached 300-1000 mm³: the test substance was administered to the latter group. The extracted tumor was placed in a cell lysate buffer (50 mM HEPES (pH 7.4), 150 mM NaCl, 10% glycerol, 1% Triton X-100, 1.5 mm MgCl₂, 1 mM EDTA, 100 mM NaF, 1 mM PMSF, 10 μg/ml aprotinin, 50 μg/ml leupeptin, 1 μg/ml peptatin A, 1 mM Na₃VO₄, 25 mM , βglycerophosphate, and phosphatase inhibitor cocktail 11) and homogenized. After centrifugation, the supernatant was protein quantified, and a 3×SDS sample loading buffer was added to prepare a cell lysate sample. Subsequently, the cell lysate was heat-treated at 94° c. for 10 minutes and cryopreserved at −20° c.

The cell lysate sample which was equivalent to 30 μg of protein was electrophoresed on a 4-20% gradient polyacrylamide gel (Daiichi Pure Chemicals Co., Ltd.). After electrophoresis, the sample was transferred to a PVDF membrane (Amersham Pharmacia Biotech Inc.) for 3 hours. In order to assay phosphorylated c-Kit, c-Kit and β-actin, immunoblot was performed using a phospho-c-kit (Tyr719) antibody (Cell Signaling Technologies, Inc.), an anti c-Kit antibody (Cell Signaling Technologies, Inc.) and an anti β-actin antibody (Sigma) as a primary antibody and an anti-rabbit IgG, HRP-linked antibody (Cell Signaling Technologies, Inc.) as a secondary antibody. After the membrane was washed, it was developed with a Super Signal (Pierce Biotechnology, Inc.).

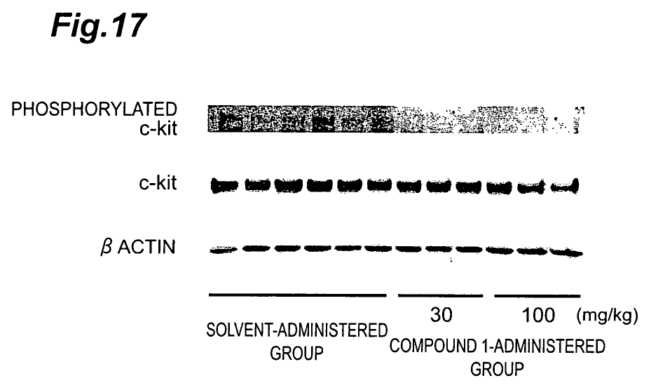

As the results are shown in FIG. 17, 4-(3-chloro-4-(cyclopropylaminocarbonyl)aminophenoxy)-7-methoxy-6-quinolinecarboxamide reduced phosphorylated c-Kit in tumor tissue when administered at 30 or 100 mg/kg, but c-Kit and β-actin remained unchanged. While 4-(3-chloro-4-(cyclopropylaminocarbonyl)aminophenoxy)-7-methoxy-6-quinolinecarboxamide completely inhibited phosphorylation when administered at 30 or 100 mg/kg, STI571 known as a c-Kit kinase inhibitor partially inhibited phosphorylation when administered even at 160 mg/kg.

These results demonstrated that 4-(3-chloro-4-(cyclopropylaminocarbonyl)aminophenoxy)-7-methoxy-6-quinolinecarboxamide inhibits phosphorylation of c-Kit in vivo, and it was confirmed that 4-(3-chloro-4-(cyclopropylaminocarbonyl)aminophenoxy)-7-methoxy-6-quinolinecarboxamide inhibits activity of c-Kit kinase and shows anti-tumor activity.

### INDUSTRIAL APPLICABILITY

As described above, the present invention can provide novel crystals of 4-(3-chloro-4-(cyclopropylaminocarbonyl)aminophenoxy)-7-methoxy-6-quinolinecarboxamide (polymorph (A) and (B)) and a process for the preparation of the same.

1. A polymorph (A) of 4-(3-chloro-4-(cyclopropylaminocarbonyl)aminophenoxy)-7-methoxy-6-quinolinecarboxamide, having a diffraction peak at a diffraction angle (2θ±0.2°) of 15.75° in a powder X-ray diffraction.

2. The polymorph (A) according to claim 1, wherein the polymorph further has diffraction peaks at diffraction angles (2θ±0.2°) of 9.98° and 11.01° in a powder X-ray diffraction.

3. (canceled)

4. The polymorph (A) according to claim 1 or 2, wherein the polymorph has an absorption band at a wavenumber of 3452.3±2.5 cm⁻¹ in an infrared absorption spectrum in potassium bromide.

5. The polymorph (A) according to claim 1 or 2, wherein the polymorph further has an absorption band at a wavenumber of 1712.2±1.0 cm⁻¹.

6. A polymorph (B) of 4-[3-chloro-4-(cyclopropylaminocarbonyl)aminophenoxy]-7-methoxy-6-quinolinecarboxamide, having a diffraction peak at a diffraction angle (2θ±0.2°) of 21.75° in a powder X-ray diffraction.

7. The polymorph (B) according to claim 6, wherein the polymorph further has diffraction peaks at diffraction angles (2θ±0.2°) of 12.430° and 16.56° in a powder X-ray diffraction.

8. (canceled)

9. The polymorph (B) according to claim 6 or 7, wherein the polymorph has an absorption band at a wavenumber of 1557.6±1.0 cm⁻¹ in an infrared absorption spectrum in potassium bromide.

10. The polymorph (B) according to claim 6 or 7, wherein the polymorph further has an absorption band at a wavenumber of 1464.4±1.0 cm⁻¹.

11. A process for the preparation of the polymorph (A) of 4-[3-chloro-4-(cyclopropylaminocarbonyl)aminophenoxy]-7-methoxy-6-quinolinecarboxamide according to claim 1, comprising a step of dissolving 4-[3-chloro-4-(cyclopropylaminocarbonyl)aminophenoxy]-7-methoxy-6-quinolinecarboxamide in a good organic solvent, followed by rapid admixing with a poor solvent.

12. A process for the preparation of the polymorph (A) of 4-[3-chloro-4-(cyclopropylaminocarbonyl)aminophenoxy]-7-methoxy-6-quinolinecarboxamide according to claim 1, comprising a step of dissolving 4-[3-chloro-4-(cyclopropylaminocarbonyl)aminophenoxy]-7-methoxy-6-quinolinecarboxamide in a good organic solvent with stirring, followed by admixing with a poor solvent in such a way that the resultant crystals precipitate when the stirring is stopped.

13. A process for the preparation of the polymorph (A) of 4-[3-chloro-4-(cyclopropylaminocarbonyl)aminophenoxy]-7-methoxy-6-quinolinecarboxamide according to claim 1, comprising a step of reacting 7-methoxy-4-chloro-quinoline-6-carboxamide with 1-(2-chloro-4-hydroxyphenyl)-3-cyclopropylurea in the presence of a base in a good organic solvent for 4-[3-chloro-4-(cyclopropylaminocarbonyl)aminophenoxy]-7-methoxy-6-quinolinecarboxamide, followed by rapid admixing with a poor solvent for 4-[3-chloro-4-(cyclopropylaminocarbonyl)aminophenoxy]-7-methoxy-6-quinolinecarboxamide.

14. The process for the preparation according to any one of claims 11 to 13, wherein the poor solvent is admixed rapidly within 10 minutes.

15. A process for the preparation of the polymorph (B) of 4-[3-chloro-4-(cyclopropylaminocarbonyl)aminophenoxy]-7-methoxy-6-quinolinecarboxamide according to claim 6, comprising a step of dissolving 4-[3-chloro-4-(cyclopropylaminocarbonyl)aminophenoxy]-7-methoxy-6-quinolinecarboxamide in a good organic solvent, followed by slow admixing with a poor solvent.

16. A process for the preparation of the polymorph (B) of 4-[3-chloro-4-(cyclopropylaminocarbonyl)aminophenoxy]-7-methoxy-6-quinolinecarboxamide according to claim 6, comprising a step of dissolving 4-[3-chloro-4-(cyclopropylaminocarbonyl)aminophenoxy]-7-methoxy-6-quinolinecarboxamide in a good organic solvent while stirring, followed by admixing with a poor solvent in such a way that the resultant crystals diffuse when the stirring is stopped.

17. A process for the preparation of the polymorph (B) of 4-[3-chloro-4-(cyclopropylaminocarbonyl)aminophenoxy]-7-methoxy-6-quinolinecarboxamide according to claim 6, comprising a step of reacting 7-methoxy-4-chloro-quinoline-6-carboxamide with 1-(2-chloro-4-hydroxyphenyl)-3-cyclopropylurea in the presence of a base in a good organic solvent for 4-[3-chloro-4-(cyclopropylaminocarbonyl)aminophenoxy]-7-methoxy-6-quinolinecarboxamide, followed by slow admixing with a poor solvent for 4-[3-chloro-4-(cyclopropylaminocarbonyl)aminophenoxy]-7-methoxy-6-quinolinecarboxamide.

18. The process for the preparation according to any one of claims 15 to 17, wherein the poor solvent is admixed slowly in 1 hour or more.

19. A process for the preparation of the polymorph (B) of 4-[3-chloro-4-(cyclopropylaminocarbonyl)aminophenoxy]-7-methoxy-6-quinolinecarboxamide according to claim 6, comprising a step of heating a polymorph (A) of 4-[3-chloro-4-(cyclopropylaminocarbonyl)aminophenoxy]-7-methoxy-6-quinolinecarboxamide, having a diffraction peak at a diffraction angle (2θ±0.2°) of 15.75° in a powder X-ray diffraction, in suspension in a mixed solvent of a good organic solvent for the polymorph and a poor solvent for the polymorph.

20. The process for the preparation according to claim 19, wherein the polymorph (A) is a polymorph further having diffraction peaks at diffraction angles (2θ±0.2°) of 9.98° and 11.01° in a powder X-ray diffraction.

21. A process for the preparation of the polymorph (B) of 4-[3-chloro-4-(cyclopropylaminocarbonyl)aminophenoxy]-7-methoxy-6-quinolinecarboxamide according to claim 6, comprising a step of heating a polymorph (A) of 4-[3-chloro-4-(cyclopropylaminocarbonyl)aminophenoxy]-7-methoxy-6-quinolinecarboxamide, having an absorption band at a wavenumber of 3452.3±2.5 cm⁻¹ in an infrared absorption spectrum in potassium bromide, in suspension in a mixed solvent of a good organic solvent for the polymorph and a poor solvent for the polymorph.

22. The process for the preparation according to claim 19 or 20, wherein the polymorph (A) is a polymorph having an absorption band at a wavenumber of 3452.3±2.5 cm⁻¹ in an infrared absorption spectrum in potassium bromide.

23. The process for the preparation according to claim 22, wherein the polymorph (A) is a polymorph further having an absorption band at a wavenumber of 1712.2±1.0 cm⁻¹.

24. The process for the preparation according to any one of claims 11 to 13, 15 to 17 or 19 to 21, wherein the good organic solvent is dimethylsulfoxide, dimethylimidazolidinone, 1-methyl-2-pyrrolidinone, N,N-dimethylformamide, N,N-dimethylacetamide, acetic acid, sulforane, or a mixed solvent of at least two of the foregoing.

25. The process for the preparation according to any one of claims 11 to 13, 15 to 17 or 19 to 21, wherein the poor solvent is water, acetone, acetonitrile, ethyl acetate, isopropyl acetate, methanol, ethanol, n-propanol, isopropanol, or a mixed solvent of at least two of the foregoing.

26. The process for the preparation according to claim 13 or 17, wherein the base is potassium t-butoxide, cesium carbonate or potassium carbonate.

27-55. (canceled)
